(19) United States
(12) Patent Application Publication
(10) Pub. No.: US 2025/0331952 A1
(43) Pub. Date: Oct. 30, 2025

(54) SYSTEM AND METHOD FOR ORTHODONTIC APPLIANCE DELIVERY

(71) Applicant: Hisham BADAWI, Calgary (CA)

(72) Inventor: Hisham BADAWI, Calgary (CA)

(21) Appl. No.: 18/866,989

(22) PCT Filed: May 19, 2023
(86) PCT No.: PCT/CA2023/050701

§ 371 Date: Nov. 18, 2024

Related U.S. Application Data
(60) Provisional application No. 63/343,730, filed on May 19, 2022.
(60) Provisional application No. 63/343,728, filed on May 19, 2022.

(51) Int. Cl. A61C 7/00; G16H 20/30

(52) U.S. Cl. CPC A61C 7/002; G16H 20/30

(57) ABSTRACT

Systems, methods, electronic devices and computer-readable media for orthodontic appliances include: generating final positions for each of the plurality of teeth represented in an orthodontic data set, including restricting the adjustments to the initial position of each tooth to adjustments having a centre of rotation substantially about the root apex point of the respective tooth; generating a series of intermediate teeth positions between the initial positions for each of the plurality of teeth represented in the first orthodontic data and final positions for each of the plurality of teeth, the series of intermediate positions comprising at least part of a treatment plan; and generating data from which orthodontic appliances, which facilitate movement through the intermediate tooth positions, can be produced.

### CROSS-REFERENCE TO RELATED APPLICATIONS

The present disclosure claims all benefit including priority to U.S. Provisional Patent Application 63/343,730, and to U.S. Provisional Patent Application 63/343,728; both of which were filed on May 19, 2022, and are entitled “SYSTEM AND METHOD FOR ORTHODONTIC APPLIANCE DELIVERY”. Both of these documents are hereby incorporated by reference in their entireties.

### FIELD

Aspects of the present disclosure relate to the field of orthodontic applications; and more particularly, some embodiments of the present disclosure relate to the field of designing orthodontic appliances for use in orthodontic treatments.

### BACKGROUND

Orthodontic tooth movement is essentially bone and soft-tissue remodeling produced with the application of a carefully designed force system acting on a tooth, this causes the tooth to be displaced relative to its supporting bony socket, thereby producing periodontal ligament (PDL) compression and tension areas. With the applied force system and subsequent displacement of the tooth, the PDL under pressure or tension is responsible for the necessary remodeling of the surrounding bone with resorption on compression areas and apposition on tension areas of the tooth's root system, producing a net change in the position of the tooth's root and clinical crown. Efficient control of the direction and magnitude of the force system applied to the clinical crown forms the foundation of orthodontic tooth movement.

Orthodontic appliances evolved as force delivery systems over more than a century, early to mid-twentieth century the fixed appliance (FA) technology was developed and popularized in the United States to replace the early removable appliance (RA) method. The standard edgewise system in turn, evolved into the preadjusted edgewise system which became the default method for delivering orthodontic tooth movement. These fixed systems were produced with a wide variety of deterministic orthodontic prescriptions of the desired final relative crown positions.

The biomechanics of the preadjusted fixed appliance system requires bonding a fixed attachment on the tooth's clinical crown with a dimensional lumen (bracket slot) which receives a dimensional archwire giving the clinician 3D control of the tooth position.

### SUMMARY

In some situations, aspects of the present disclosure may enable the generation of orthodontic appliances and treatment plans with limited or no use of fixed/bonded appliances.

In some situations, aspects of the present disclosure may enable the generation of orthodontic applications and treatment plans with limited or no need to interproximal enamel reduction.

In various aspects, the present disclosure provides systems, methods, electronic devices and computer-readable media for orthodontic appliance delivery.

In various aspects, the present disclosure may, in some situations, provide systems, methods, electronic devices and computer-readable media for orthodontic treatment and treatment planning.

In accordance with one aspect, there is provided a computer-implemented method for orthodontic appliances. The method includes: obtaining from a storage device or generating a first orthodontic data set representing initial positions for each of a plurality of teeth in at least a portion of a dentition, the first orthodontic data set including a position of a root apex point for each of the plurality of teeth; generating, using the first orthodontic data set, a user interface for receiving, from at least one input device, at least one input to adjust an initial position of a first tooth of the plurality of teeth to a second position; wherein the user interface restricts the adjustments to the initial position of the first tooth to adjustments having a centre of rotation substantially about the root apex point of the first tooth; adjusting the data representing the first orthodontic data set based on the at least one input to include data representing a final position of the first tooth; generating a series of intermediate teeth positions between the initial positions for each of the plurality of teeth represented in the first orthodontic data and final positions for each of the plurality of teeth, the series of intermediate positions comprising at least part of a treatment plan; and generating signals for outputting at least a portion of the series of intermediate positions.

In some of the above embodiments, generating the signals for outputting at least the portion of the series of intermediate positions includes generating data from which 3D models for orthodontic appliances can be produced; the orthodontic applications for facilitating physical movement of the plurality of teeth from the initial positions to the final positions.

In some of the above embodiments, the based on the series of intermediate teeth positions, the 3D models are generated which enable the orthodontic appliances facilitate physical movement of the plurality of teeth without engaging with any attachment appliances on the plurality of teeth.

In some of the above embodiments, the wherein generating the signals for outputting least the portion of the series of intermediate positions includes signals for displaying a visual representation of at least a portion of the series of intermediate positions on a display, or storing data representing at least the portion of the series of intermediate positions on a storage device.

In some of the above embodiments, the root apex point for at least one tooth of the plurality of teeth is within 3 mm of a root apex of the at least one tooth in the first orthodontic data.

In some of the above embodiments, the root apex point for at least one molar of the plurality of teeth is at a distal pivot archform point as viewed on the transverse plane.

In some of the above embodiments, the user interface is configured to restrict the adjustments to the initial position of the first tooth to adjustments having a centre of rotation substantially about the root apex point of the first tooth by displaying user interface elements representing the first tooth position moving substantially about the root apex point of the first tooth in response to received inputs.

In some of the above embodiments, the user interface is configured to restrict the adjustments to the initial position of the first tooth to adjustments having a centre of rotation substantially about the root apex point of the first tooth by: allowing the first tooth position to be intermediately translated or rotated in directions or along axes which are not restricted to adjustments having the centre of rotation substantially about the root apex point of the first tooth; and generating signals for the user interface restricting the acceptance of a second tooth position defined by the adjustments for the first tooth as a final position for the first tooth when a total adjustment of the first tooth from the first position to the second position does not have a centre of rotation substantially about the root apex point of the first tooth.

In some of the above embodiments, the user interface is configured to restrict the first tooth of the plurality of teeth to a molar.

In some of the above embodiments, the user interface is configured to restrict the first tooth of the plurality of teeth to a second molar or a rear molar.

In some of the above embodiments, the method includes generating, using the first orthodontic data set, a user interface for receiving, from the at least one input device, a tooth selection input for selecting the first tooth for which the initial position is to be adjusted; wherein the user interface is configured to restrict the tooth selection of the first tooth to a molar.

In some of the above embodiments, the method includes generating, using the first orthodontic data set, a user interface for receiving, from the at least one input device, a tooth selection input for selecting a subsequent tooth for which the initial position is to be adjusted; wherein the user interface is configured to restrict the tooth selection for the subsequent tooth to be an anteriorly adjacent tooth to a previously adjusted tooth.

In some of the above embodiments, the method includes generating, using the first orthodontic data set, a user interface for receiving, from the at least one input device, a tooth selection input for selecting a subsequent tooth for which the initial position is to be adjusted; wherein the user interface is configured to restrict the tooth selection such that adjustment of the plurality of teeth progresses from the posterior to the anterior of the dentition.

In some of the above embodiments, the method includes generating, using the first orthodontic data set, a user interface for receiving, from the at least one input device, a tooth selection input for selecting a subsequent tooth for which the initial position is to be adjusted; wherein the user interface is configured to restrict the tooth selection such that adjustment of a tooth of the plurality of teeth situated in an upper jaw of the dentition is adjusted before a corresponding tooth in the lower jaw of the dentition.

In some of the above embodiments, the method includes determining, using the first orthodontic data set, a biologic archwidth of the dentition.

In some of the above embodiments, the biologic archwidth is based on based on a distance between second molars, or a distance between terminal molars.

In some of the above embodiments, the method includes the user interface is configured to restrict the adjustments to the initial position of the first tooth to adjustments based on the biologic archwidth.

In accordance with another aspect, there is provided a method for generating an orthodontic treatment plan with a computing device, the method including: inputting, with at least one input device, for each tooth in a plurality of teeth in an orthodontic data set representing at least a portion of a dentition, at least one input to adjust an initial position of each tooth of the plurality of teeth to a second position, wherein the adjustments to the initial position of each tooth has a centre of rotation substantially about the root apex point of the tooth; generating, the computing device, a series of intermediate teeth positions between the initial positions for each of the plurality of teeth represented in the orthodontic data and final positions for each of the plurality of teeth, the series of intermediate positions comprising at least part of a treatment plan; and generating signals for outputting at least a portion of the series of intermediate positions.

In accordance with another aspect, there is provided an electronic device for charting dental information, the device includes: at least one memory; and at least one processor configured for performing any of the methods described above or herein.

In accordance with another aspect, there is provided a non-transitory, computer-readable medium or media having stored thereon computer-readable instructions which when executed by at least one processor configure the at least one processor for: performing any of the methods described above or herein.

In accordance with another aspect, there is provided a method for orthodontic appliances, the method includes: obtaining or generating a first orthodontic data set representing initial positions for each of a plurality of teeth in at least a portion of a dentition, the first orthodontic data set including a position of a root apex point for each of the plurality of teeth; generating, using the first orthodontic data set, final positions for each of the plurality of teeth represented in the first orthodontic data set; wherein generating the final positions includes restricting the adjustments to the initial position of each tooth to adjustments having a centre of rotation substantially about the root apex point of the respective tooth; generating a series of intermediate teeth positions between the initial positions for each of the plurality of teeth represented in the first orthodontic data and final positions for each of the plurality of teeth, the series of intermediate positions comprising at least part of a treatment plan; and generating data from which orthodontic appliances, which facilitate movement through the intermediate tooth positions, can be produced.

In some of the above embodiments, generating the final positions includes determining end positions for each tooth based on an optimization function.

In some of the above embodiments, generating the final positions includes utilizing a user interface which restricts the adjustments to the initial position of a first tooth to adjustments having a centre of rotation substantially about the root apex point of the first tooth.

### BRIEF DESCRIPTION OF THE DRAWINGS

By way of example, reference is now made to the accompanying drawings, in which:

FIG. 1 shows an example system suitable for orthodontic treatment, planning and/or appliance delivery.

FIG. 2 shows an example computing device or system.

FIG. 3 shows a flowchart illustrating aspects of an example method for orthodontic appliances.

FIG. 4 shows a second flowchart illustrating aspects of an example method for orthodontic appliances.

FIG. 5 shows a craniofacial model illustrating anatomic planes.

FIG. 6 shows an M-D view of teeth in an example initial archform.

FIG. 7 shows an M-D view of teeth in an example final archform.

FIG. 8 shows an M-D view of teeth in an example final archform including the shapes of the initial and final archforms.

FIG. 9 shows two example visual representations of orthodontic data illustrating a reduction in the base dentition.

FIG. 10 shows an example visual representation of a portion of a dentition with the dotted lines illustrating the tooth boundaries and arch.

FIG. 11 shows an example visual representation of a sculpted tooth.

FIG. 12 shows an example interface including visual representations of each tooth and their respective centers of rotation at the tooth's root.

FIG. 13 shows example visual representations of a portion of a dentition including interface elements showing some parameters/constraints.

FIG. 14 shows an example visual representation of a tooth, and potential rotations.

FIGS. 15, 16, 17, 18, and 19 show example visual representations of an upper arch portion of a dentition with movement annotations as they are sequentially configured via an example system/method.

FIG. 20 shows example visual representations of portions of a dentition in consideration of arch symmetry.

FIGS. 21, 22, 23, 24, 25, and 26 show example visual representations of a lower arch portion of a dentition with movement annotations as they are sequentially configured via an example system/method.

FIGS. 27 and 28 show example visual representations of portions of a dentition in consideration of arch symmetry, and inter arch mechanics.

### DETAILED DESCRIPTION

Aligners are one form of appliances which evolved from the preadjusted edgewise system where the aligners are used as a more esthetic method. This method strives to plan and produce what fixed appliances produce in terms of tooth movements. This includes using the archform (overall dimensions and shape) of the patient as an input in the treatment planning process.

Current aligner providers evolved from the preadjusted edgewise system with treatment plans largely based on the fixed appliance mechanotherapy.

The current aligner setup software solutions lack the biomechanics modelling to dictate

the type of tooth movement or the most optimal path teeth should take, it instead relies on linear approximation of the paths between limits (linear or angular) of tooth movement, which is essentially the overall delta between initial state and any other state including the final desired state, while ignoring the path of synchronized motion each tooth must take in order to realize the planned outcome. This deficiency in Cad-Cam solutions applied to clinical motion in orthodontics is the main reason for lack of predictability of aligner treatments. The default aligner process of today relies on features to address the lack of predictability: over-correction, IPR, attachments, staging, lengthy treatments and-wear patterns challenging to patients.

Aligner attachments are routinely utilized in current/conventional aligner methods, attachments are artificial tooth-colored geometries added to some or all teeth for the supposed purpose of increasing predictability of tooth movement allegedly giving the clinician three-dimensional control of the tooth including the root, in line with the mindset of fixed appliance mechanotherapy.

Inter-proximal enamel reduction IPR is a process of removing a certain thickness of enamel between two moving adjacent teeth, the purpose is to eliminate crown to crown collisions that might otherwise restricted the production of planned outcome.

Staging is a term used by aligner manufacturers to refer to a functionality intended to solve actual-expected outcome discrepancies due to crown to crown-crown collisions. Staging in the conventional aligner process is essentially prioritizing planned movements in a sequential manner to eliminate potential crown to crown collisions during treatment, producing a lengthy sequence of aligners and lengthy treatments in-turn.

In some situations, aspects of the present application reduce or eliminate the need for these features, and may increase predictability of movement towards the desired outcome.

Aspects of the present application are based on the recognition that any rotation around any point on the tooth's clinical crown is difficult to produce and unlikely to be successful, since it would require considerable changes to the tooth's root apex position and the nerves/vessels connected to it. Fixed appliance mechanotherapy is based on full 3D control of the tooth via a clinical crown attachment, which by definition requires movement of the root apex.

In some situations, aspects of the present application facilitate more natural tooth movement which minimizes any change to the root apex position and consequently to the nerves attaching to the root apex.

In order to better understand the limitations of orthodontic tooth movement, it helps to think of the dentition as calcified nerve endings despite being a gross oversimplification of a very complex anatomical structure. Any rotation around any point on the tooth's clinical crown is difficult to produce and unlikely to be successful since it would require considerable changes to the tooth's root apex position including nerves, blood vessels and trans-septal fibers connected to it. The most natural tooth movement is one that minimizes any change to the root apex position and consequently to the nerve and blood vessel bundles attaching to the root apex. Fixed appliance mechanotherapy is based on full 3D control of the tooth via a fixed clinical crown attachment, which by definition requires movement of the root apex.

In some embodiments, aspects of the orthodontic process involve the generation and/or execution of a treatment plan and/or a set of physical aligners which are sequentially attached to a dentition to facilitate tooth movement.

In some embodiments, a computer-implemented method includes: obtaining data structures and/or code which represent models of individual teeth in a dentition; generating user interface displays for receiving input to change the positions and orientations of the teeth to define a visual treatment objective; applying an interpolation process to determine a series of intermediate positions between the initial model state and the final state defined by the visual treatment objective; and generating 3D models of interpolated arch models for facilitating movement of the teeth between the intermediate positions. Orthodontic appliances, such as aligners, can be produced from these 3D models.

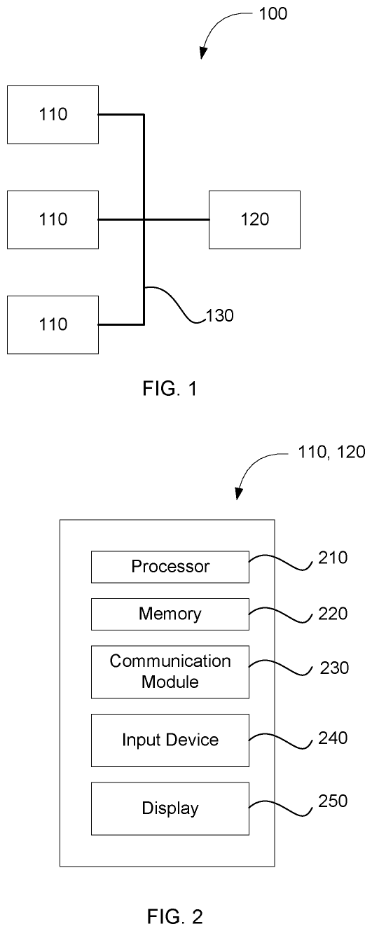

FIG. 1 shows an example system 100 for orthodontic appliance delivery. In some examples, aspects of the system can be used to help an orthodontic practitioner to create and maintain an orthodontic treatment plan on an electronic device. Aspects of the example systems can, in some examples, be a component of a larger process for producing orthodontic appliances for facilitating the treatment plan.

For the purposes of this disclosure, the term orthodontic and its variants are not limited to the context of the practice, information, and treatment planning performed by orthodontists, but can include anything related to one or more aspects of the mouth or oral treatment, including but not limited to anything related to the practice of dentistry, orthodontics, periodontics, endodontics, prosthodontics, and the like.

In the example shown, system 100 includes one or more client devices 110 on which orthodontic information can be accessed, displayed, or modified. In some examples, the client devices 110 can connect to a central device 120. The central device 120 can, in some examples, be a server or electronic database for hosting orthodontic models, treatment plans, appliance designs, and the like. It can also software applications or modules for performing various aspects of the orthodontic treatment planning and/or appliance delivery system.

For example, an orthodontic system can include several client devices 110 at different locations such as treatment rooms, reception desks, counseling areas, or offices. In some examples, the client devices 110 and central device 120 can be at different locations such as terminals in different offices or a server or database hosted at a remote location. Orthodontic models, treatment plans, appliance designs, or application-related instructions or data can be communicated between devices via network 130. The network 130 can include one or more private and/or public networks. The network 130 can include a wired network such as a wired local area network or the internet, or wireless networks such as cellular telephone networks or Wi-Fi networks.

While the example system shows three client devices and one central device, any number of client or central devices can be used in any suitable arrangement.

In some examples, the central device 120 can host or have access to a database storing orthodontic information. In some examples, the central device 120 can provide processing or host an application or software module accessible by a client device for performing various aspects of the methods described herein.

In some examples, the central device 120 itself may also be a client device 110 on which orthodontic information can be accessed, displayed or modified.

In some examples, a single electronic device 110 such as a personal computer can be used to store, access, display and modify orthodontic information.

In some examples, the system can include a database located at a client device 110, a central device 120, or elsewhere on the network. The database can, in some examples, store orthodontic information. In some examples, local or backup copies of orthodontic information can be stored at a multiple locations including a client device 110, a central device 120, or elsewhere in the system.

Examples of client 110 and central devices 120 can include, but are not limited to, computers, servers, tablet or mobile computers, or mobile phones.

FIG. 2 shows an example client 110 or central device 120. The device 110, 120 can include one or more processors 210 connected to one or more memories 220, communication modules 230, input devices 240, or displays 250. In some examples, a memory 220 can store modules which enable a processor 210 to perform any aspect of the methods described herein. In some examples, a memory 220 can store orthodontic information. In some examples, a memory 220 can store models, images, renderings or other visual representations of various anatomical structures or orthodontic appliances.

In some examples, a device 110, 120 can include a communication module 230 which may include hardware or software for communicating orthodontic or application information over network 130.

In some examples, a device 110, 120 can include or be connected to one or more input devices 240 for receiving inputs to edit orthodontic information or to otherwise operate the device 110. Input devices can include keyboards, mice, touchscreens, touchpads, navigation devices, remote controls, tablet computers, mobile phones, or other suitable input devices.

In some examples, a device 110, 120 can include or be connected to a display 250 for displaying aspects of an orthodontic treatment process.

In some examples, computer-readable instructions such as a computer program or application can be installed or otherwise operable on a client or central device, or can be stored on a non-transitory, computer-readable medium.

In some examples, when orthodontic information is stored at a central device, it can be accessed by different users at different locations or on different client devices.

In some examples, one or more devices 110, 120 can be configured to request, receive and/or verify user credentials to control access to orthodontic information stored in the system 100. In some examples, user credentials or identifiers may be stored in association with additions, deletions, or other modifications performed on orthodontic information.

The system 100, in some examples, provides a digital design environment in which aspects of the system can be used to create and store orthodontic treatment plans, aspects of a dentition, and/or appliances.

In some examples, the system can model orthodontic information for any dental procedure, including orthodontic, periodontal, endodontic, pedodontic, medical, and oral-surgical procedures.

In some examples, one or more devices 110, 120 and/or one or more of their processor(s) 210 can be configured to perform any aspect(s) of the methods described herein.

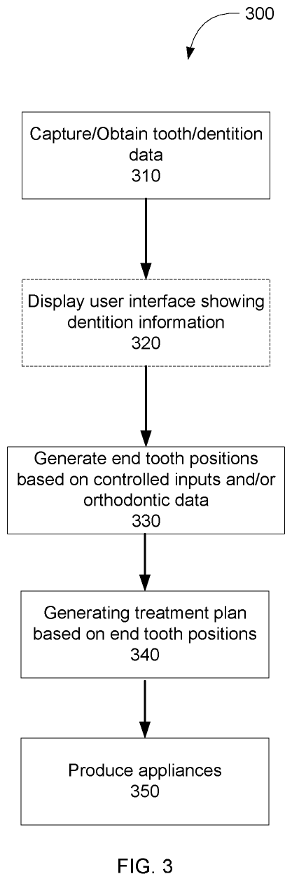

FIG. 3 shows a flowchart showing aspects of an example method 300 for orthodontic appliance delivery and/or treatment planning/execution. The flowchart herein show example operating sequences of example system 100; however, it is to be understood that aspects of different flowcharts can be combined or can be performed in any suitable order.

At 310, device(s) or a system are configured to obtain orthodontic data. In some embodiments, the orthodontic data is obtained by capturing via one or more sensors/imaging devices data representing the size, shape, surface, location, orientation, etc. of one or more anatomic or other objects in the mouth (teeth, gums, bones, appliances, etc.). In some embodiments, the dental data can be captured with imaging devices, point cloud generators, 3D scanners, depth sensors, thermal sensors, x-rays, and/or any other type of device or methodology for capturing this information.

In some embodiments, the orthodontic data set is obtained by retrieving the data from a memory or another storage location/device locally or via a network 130.

In some embodiments, the orthodontic data set includes capturing image data, point cloud data, depth data, thermal data, x-ray data and/or any other type of data representing the outer surfaces and locations of the anatomic objects.

In some embodiments, the orthodontic data set such as point cloud data for an entire or portion of a dentition can be used to obtain further orthodontic data set such as surface models for individual objects (e.g. teeth). This can, in some embodiments, include 3D surface model construction of teeth, and/or segmentation of the surface model into individual objects.

In some embodiments, the dental data can be captured with an intraoral scanner such as a 3Shape TRIOS 5, and/or multiple generations of the iTero Element. In some embodiments, the intraoral scanner projects a light onto the scanning surface(s) and captures plurality (e.g. thousands) of images which can be processed to produce an accurate digital model of the teeth and surrounding tissues. In some embodiments, a scan should capture all the teeth and surrounding gingiva without any voids.

In some embodiments, one or more processors can be configured to generate signals for displaying a graphical user interface including a visual representation of at least a portion of the dentition. In some embodiments, the processors are configured for generating a user interface for receiving, from at least one input device, at least one input to facilitate segmentation and/or identifying the areas/volumes of a particular tooth or other anatomical feature.

In some embodiments, the one or more processors can be configured to reduce the size of the orthodontic data by removing portions around the teeth which may not be required for manipulating teeth and/or for forming appliances. In some embodiments, the processor(s) provide a user interface for selecting regions to remove/retain. In some embodiments, the processor(s) are configured to automatically reduce the size of the orthodontic data based on defined, algorithmic or trained models to form a base dentition region with margins around teeth suitable for manipulation and/or application formation.

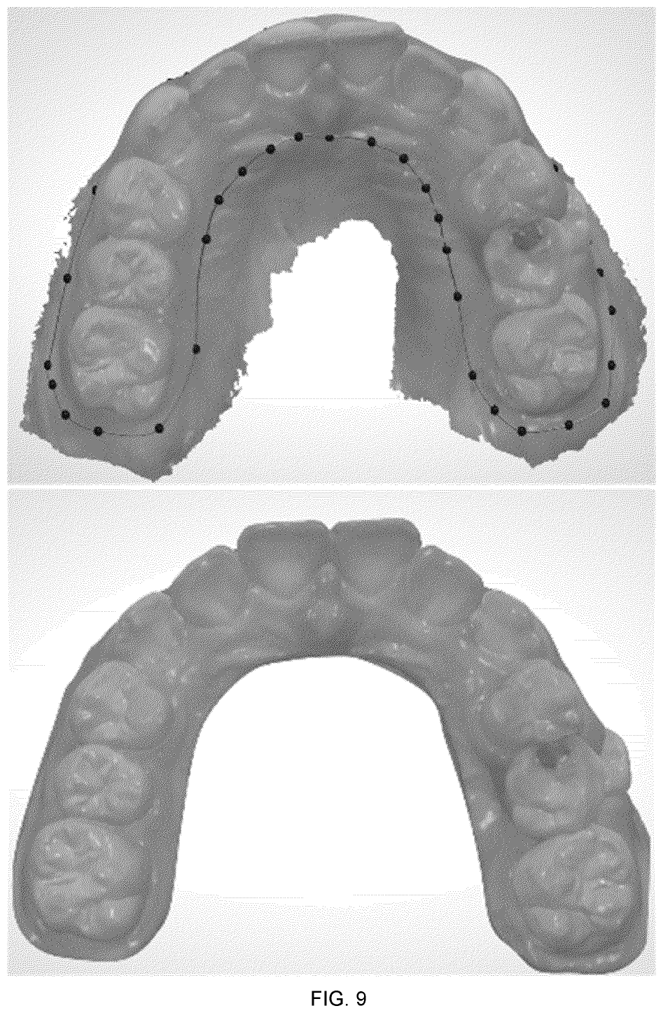

FIG. 9 shows an example user interface including a visual representation of an arch in a dentition before (top) and after (bottom) the orthodontic data has been reduced.

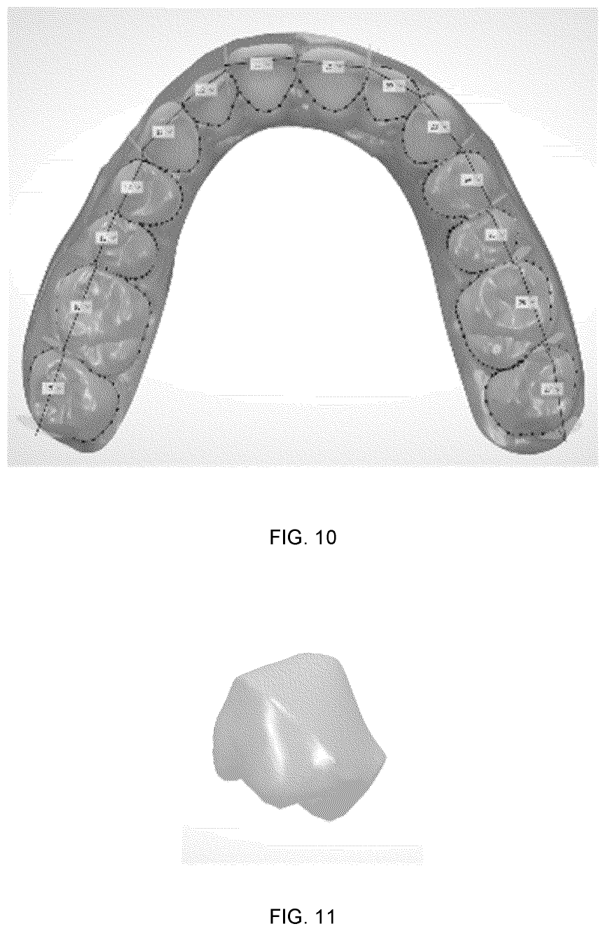

In some embodiments, the processor(s) are configured to segment individual teeth from each other. In some embodiments, this process is performed automatically based on the dentition data. In some embodiments, the process can be manual with the processor(s) providing a user interface for receiving inputs to define the boundaries of the teeth. In some embodiments, the processor(s) can automatically generate boundaries and can provide a user interface for receiving inputs to refine these boundaries. FIG. 10 shows an example visual representation of a portion of a dentition with the dotted lines illustrating the tooth boundaries and arch.

In some embodiments, the processor(s) are configured to virtually sculpt the individual teeth. This is the process by which any sharp edges or corners on the surface of the tooth are smoothed out, ensuring that the final model produced is an accurate representation of the patient's teeth. FIG. 11 shows an example visual representation of a sculpted tooth.

In some embodiments, any suitable capturing device(s), data processing step(s) and/or any combination thereof which generate data representing the shape/surfaces of teeth and their relative locations can be used.

The orthodontic data set, in some examples, can include parameters defining models of teeth, implements, gums, restorations, fractures or other objects in a patient's mouth. In some examples, these models can define relative dimensions and boundaries of the object. The models can also, in some examples, define rules/limits/constraints of how the object can be scaled, moved, rotated, be attached or interact with other objects. In some examples, models can be stored as part of a software package or a library on a client or central device.

In some embodiments, obtaining the orthodontic data set can include any number of steps as described herein or otherwise, and can include all or a subset of the following: the capturing of image or other data from a patient's dentition with one or more input devices, the transmission/communication/storage of the captured data, the processing of the image data or any other function to convert, translate, manipulate, or otherwise process the data into a format usable by one or more processors and/or users to perform the methods provided herein.

In some embodiments, the orthodontic data set includes parameters or sets of values defining tooth dimensions, sizes, surfaces, locations and/or the like. In some embodiments, the orthodontic data set can be defined by data structures or logic which associate or group data values by tooth type. For example, different data structures or fields can be defined to store values which indicate a type of tooth (e.g. this could include a tooth category/group e.g. a molar, an incisor, a canine, pre-molar, etc.; or a specific tooth type, e.g. 1st molar, central incisor, etc.), a category of tooth (e.g. primary (baby) or permanent (adult) tooth), a relative location of a tooth (e.g. upper vs. lower arch, 1^{st} vs. 2^{nd} molar, etc.) and the like.

In some embodiments, tooth data structures can be instantiated with different fields or different values based on different models or data class/structures associated with a particular tooth type.

In some embodiments, different tooth types can be subject to different rules, dimension ranges/limitations, and/or manipulation/motion types/ranges/limitations. For example, a molar may have different possible manipulations from an incisor. In some embodiments, these rules can be defined in a data structure (e.g. Java™ class) or can be defined in the computer instructions controlling the handling of user input.

In some embodiments, the different tooth types can have different anatomical location/dimension fields and/or rules. For example, an incisor or canine will typically have a single root, while molars will typically have two or three. Accordingly, the data structures and/or computational logic for defining and/or handling calculations involving anatomical landmarks or other tooth specific data such as a root apex location can vary based on the type of tooth.

In some embodiments, the data structures and/or computational logic can include data and/or fields for each specific tooth in a human dentition. For example, the data structures can be configured to store or have fields for storing data for each particular tooth (e.g. data structures/fields for upper left central incisor, data structures/fields for upper right central incisor, data structures/fields for upper left lateral incisor, . . . , etc.). In some embodiments, there can be a dedicated data structure for storing the parameters for each possible tooth.

In some embodiments, the same data structure(s) can each be used for multiple teeth with field(s) which specify the particular tooth in the dentition. For example, a data structure can have standard fields for storing dimension parameters which could be applicable to many types of teeth, and at least one field which identifies the particular tooth in the dentition (e.g. a field storing a value which identifies the tooth as an upper left 1^{st} bicuspid. This could be a number, an enumerated value or any other value(s). e.g. 24 FDI notation, International Tooth Numbering System #12).

In some embodiments, the data structures can include data representing location data which can be used to identify a tooth's location in 3-dimensional space.

In some embodiments, the orthodontic data set and/or tooth data structures can be defined in any manner which enables a processor and/or user to generate a visual representation of or otherwise visualize the teeth in a dentition. In some embodiments, this visual representation/visualization can include the teeth's sizes, surfaces, and locations relative to one or more other teeth in the dentition.

In some embodiments, the orthodontic data set includes surface, size, position, orientation and/or landmark information regarding the root(s) of teeth.

In some embodiments, the orthodontic data set includes centers of rotations for each tooth. In some embodiments, the centers of rotation are adjusted to approximate the apical extent of the tooth's root. In some embodiments, the processor(s) are configured to define this center of rotation based on orthodontic data such as x-ray images, and/or from user inputs received via a user interface.

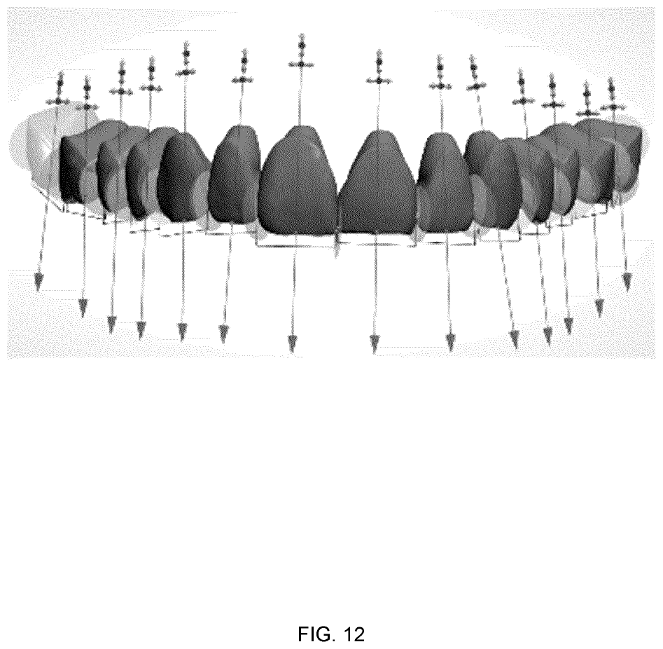

FIG. 12 shows an example interface including visual representations of each tooth and their respective centers of rotation at the tooth's root.

At optional 320, one or more processors can be configured for generating signals for displaying, on a display, a visual representation of at least a portion of a dentition represented by the orthodontic data set. In some examples, this visual representation can be updated in real-or near real-time as changes are made to an orthodontic data set and the underlying parameters.

In some examples, displaying the visual representation can include displaying visual representations of objects in a dentition. In some examples, the objects, such as teeth, gums or other anatomical aspects, and/or implements, can be displayed based on the associated parameters in the orthodontic data set. In some examples, the visual representations can include dental conditions, restorations or dental appliances on one or more teeth based on the parameters of the orthodontic data set.

In some embodiments, the visual representation can be generated based on numerical surface values and/or formulae defining the shape, dimensions and/or location of the objects.

In some embodiments, the visual representation and/or other aspects of the present disclosure can be provided by a CAD/CAM (computer-aided design/computer-aided manufacture) software or other code providing similar functionality being executed on the processors.

In some embodiments, instead of or in addition to displaying a visual representation of the dentition, processors can be configured to display a user interface with numerical values representing the orthodontic data set.

With initial orthodontic data set for defining, representing and/or visualizing each tooth and its relative position in the dentition, a digital treatment plan for physically manipulating the teeth to a final desired position can be created. In some situations, this can be considered an outcome plan which defines a desired final state of all tooth positions (and not necessarily the precise movements from the positions defined in the initial orthodontic data set to the final state). In some environments, this is referred to as “digital treatment planning” or orthodontic outcome prediction using 3D digital tools for setting Visual Treatment Objectives (VTO).

One approach to aligner treatment planning includes:
i. Point cloud acquisition of the dental arch anatomic objects (e.g. teeth)
ii. 3D surface model construction of teeth
iii. Segmentation of the surface model into individual objects
iv. Orthodontic movement planning using CAD/CAM
v. Timepoints of linear interpolation selected to produce interpolated arch models
vi. 3D printing of the interpolated arch models, or direct printing of aligners
vii. Production of thermoplastic formed clear appliances (also known as aligners)

As discussed above with respect to 310, steps i, ii, and iii above can be captured by imaging devices, and generated by one or more processors configured to compute surface models of individual teeth.

In iv, traditional orthodontic CAD/CAM software enables the freedom of an operator to move a tooth in any direction into a desired final position. In some instances, the traditional approach may restrict the absolute magnitude of a movement but not the direction of the movement. In some situations, using one or more phases of this unrestrained orthodontic treatment planning approach, enable a user to move teeth to any final desired position.

In some embodiments, aspects of the present disclosure define and/or enforce the restriction of final tooth positions in accordance with a biomechanics architecture for the optimization of root movements and/or the synchronization of relative crown movement. In some instances, this can facilitate desired natural tooth movements which can in some situations reduce treatment times, reduce the number of appliances and/or reduce or completely eliminate the need for filing of teeth to facilitate desired tooth movement.

In some instances, the part of the aligner corresponding to a segmented tooth can be referred to as aligner cap. In some embodiments, the systems and methods described herein utilize or facilitate LaGrangian paths convex optimization of relative clear aligner cap positions which can, in some situations, produce least action (least time) force system therapy.

In some embodiments, the systems and methods described herein can utilize commercially available aligners to produce biomechanically-optimized, minimally-invasive clear aligner therapy. In some embodiments, the aligners are designed along with a treatment plan to utilize normal functional occlusion to produce suctional aligner negative pressures guiding the dentition to follow the predetermined optimized paths towards the most optimum final state, which in some embodiments factors for tongue volume changes during orthodontic therapy.

In addition to convex optimization, the systems and methods described herein may utilize the physics of clinical orthodontic philosophy translated into the language of mathematics, and LaGrangian mechanics using the language of calculus of variations. LaGrangian mechanics eliminates many of the limitations of Newtonian mechanics reformulating problems without the use of vector notation of Newtonian mechanics, which is based on defining free body diagram vectors to form equations of motion. There are numerous problems that are almost impossible to analyze with Newtonian mechanics, orthodontics mechanics is one.

Most orthodontic problems result from the functional imbalance between forces acting on the external (buccal or labial) surfaces of teeth and opposite forces acting on the internal (lingual/palatal) surfaces of teeth. This imbalance over time produces what we refer to as the Collapsed Arch Syndrome (CAS).

Performing full arch simultaneous biomechanically optimized tooth movements is critical to solving CAS using aligners and without the usual outcome deficiencies of standard aligner treatments, including open bite tendencies and protruded or crowded dentition. The implementation of full arch simultaneous biomechanically optimized movements which in some situations may produce considerable improvement in the quality of outcomes with overall treatment time reduction and/or TMD (temporomandibular disorder) or periodontal benefits.

In some embodiments, the systems and methods described herein utilize, facilitate and/or enforce simultaneous counterbalanced rotation vectors, which in turn produce specific LaGrangian paths of clinical crowns, caused by synchronized rotations of teeth to produce the most natural archform for any given patient, resolve malocclusion and maximize tongue volume within the patient specific and general dentofacial constraints. In some embodiments, the systems and methods provide an aligner protocol which can produce simultaneous synchronized relative crown movements that can satisfy the necessary restrictions on root apex movements by following biomechanically constrained LaGrangian paths that resolve the malocclusion while normalizing tongue volume.

In some embodiments, the systems and methods can facilitate the reconstruction of the overall shape of the dental arch restoring the “natural” relative clinical crown orientations and positions. In some embodiments, the systems and methods facilitate this by creating an aligner treatment plan setup that produces net simultaneous moments on all teeth in the dental arch with an aim to produce rotations around predetermined centers of rotation, one per individual tooth. These simultaneous moments are counter-balanced between the right and left sides. In some instances, the outcome is a minimally invasive method capable of applying personalized biologically sensible forces and moments, producing dental movements in line with the nature of dental biomechanics, and constrained by the rest of the craniofacial biomechanical assembly of functional objects in equilibrium. The net outcome is differential expansion or arch development force and moment vectors that deliver the VTO planned using the aligner protocol and dental arch reconstruction method described herein.

In some embodiments, the systems and methods described herein facilitate or enforce constraints on the root and crown system which can help ensure the predictability, reliability, or effectiveness of the path of motion of every clinical crown required to achieve a predetermined VTO.

In some embodiments, the systems and methods described herein utilize an optimization metric, cost or reward function when deciding between different treatment/movement options. In some embodiments, when faced with an option, the systems and methods can select or identify an option which maximizes a given metric and/or minimizes a cost. In some embodiments, to follow a traditional cost minimization convention, the reward (e.g. maximizing tongue volume) can be multiplied by minus one to convert a maximization problem to a mathematical minimization one.

For a tooth, there are two main vectors of motion with infinite possibilities for each: a translation vector and a rotation vector. In some embodiments, the system/methods are configured and/or defined to constrain the location of the center of rotation and to disallow or minimize translation. In some embodiments, with these constraint(s), a value x is selected from the continuum of all real numbers that:
Maximizes the tongue volume function F by one rotation around the tooth's designated center of rotation;
Satisfies the local coordinate constraints;
Moves M-D (mesial-distal) points closer to archform of most convexity

In some embodiments, the systems and methods described herein utilize terminal molar rotation to define the path of the anterior dentition through a prioritized rotation optimization process that can achieve a mathematical convex function of arch form geometry.

In some embodiments, the systems and methods described herein implement or facilitate orthodontic optimization based on some or all of the following aspects:
a set of potential decision variables: e.g. translation and rotational movement variables (T,R) for N teeth (28 for standard adult dentition)
cost function F which includes a tongue volume factor to be maximized. In some instances, this can also be expressed in planar terms. This function can map the set where our decision variables live, it is sometimes referenced as the objective function.
decision variables. The equality and inequality constraints together define the feasible set or the set of possibilities from which decision variables x, y, z can be selected.

In some embodiments, F is a Cubic function (arch depth height, width) and archform area is a quadratic function of M-D line segments integration. The functions that define the constraints are nonlinear functions resulting in a nonlinear problem.

The orthodontic equations of motion can be reformulated to eliminate the cumbersome vectorial system that is used in Newtonian mechanics and instead use the Hamilton principle of least action or least time.

$$S = {\int }_{{t}_{1}}^{{t}_{2}} L ( {q}_{i} , {\dot{q}}_{i} , t )$$

The integral over a time period of some functionality L which is defined in terms of some dentofacial coordinates for every tooth, where q; represents all the coordinates that are needed to define the tooth in the craniofacial/dentofacial system. From Hamilton's principle of least action, it can be seen that stationary points to this equation are to be found.

In some embodiments, L is defined as a function to be minimized to find stationary points in accordance with Hamilton's principle which produce the path of least action that the dentofacial coordinates are going to take from initial state to final state coordinates. Once this minimization is applied to it, the outcome can conform to the path that satisfies Newton's second law, conservation of energy, conservation of mass, conservation of momentum. In some embodiments, L is defined such that it provides the dentofacial paths that conform to laws of physics and constraints referenced herein.

To derive this function L, which is an unknown right now, such that we get the appropriate equations of motion for our orthodontic problem using the Method of Virtual Work approach to provide us with the correct form of this function when we find the stationary points to it. When we apply the Euler-LaGrange equation we can derive the equations of motion or paths that our dentofacial system evolves to, which conforms to Newton's second law, conservation of energy, momentum, and mass.

The function L is defined as the LaGrangian, and can be stated as:

$$L ( {q}_{i} , {\dot{q}}_{i} , t ) = T - U$$

T=The kinetic energy of the system

$$T = \frac{1}{2} m ( {\dot{x}}^{2} + {\dot{y}}^{2} + {\dot{z}}^{2} )$$

U=The potential energy of the system,

Calculated using virtual work method, using

$$U = U ( \vec{r} ) = U ( x , y , z ) or U ( r , θ , z )$$

The LaGrangian scalar quantity is a quantity that can be chosen such that the stationary points according to Hamilton's principle converge to the paths that Newton's second law and conservation laws dictate.

The LaGrangian is going to be defined in terms of the coordinates qi for this system, and an equation can be defined for each one of the qi coordinates of every point P of interest in the mesio-distal convex sets (M-D convex set) in the dentofacial space. The calculus of variations can be used to minimize the LaGrangian function for every qi for every tooth, to find stationary points q using Hamilton's principle of least action.

Differentiation of the function L with respect to the path variable Y has to be equal to the derivative of the independent variable t, of the derivative of the function L with respect to the derivative of the path variable:

$$\frac{δ L}{δ q} = \frac{d}{{d}_{t}} ( \frac{δ L}{δ \dot{q}} )$$

Where,

$$\frac{d}{dt} S = {\int }_{{x}_{1}}^{{x}_{2}} L ( q , \dot{q} , t ) = {\int }_{{y}_{1}}^{{y}_{2}} L ( q , \dot{q} , t ) = {\int }_{{z}_{1}}^{{z}_{2}} L ( q , \dot{q} , t ) = 0$$

Suppose there is some point P within the Convex Set M-D of a tooth within the upper archform of the craniofacial domain from the origin O. The position of this point at any period of time can be defined as a vector r in order to find where it is in space, where:

$$\vec{r} = x \hat{ι} + y \hat{J} + z \hat{k}$$

To find the equation of motion for P without using any kind of Newtonian mechanics, using instead the Euler-LaGrange equation to arrive at what is known as the LaGrangian L for the qi variable, the independent variable is time (t) and for every period of time, there are different x, y and z coordinates of point P within the M-D convex set, which will eventually give the 3D LaGrangian path variable. In other words, defining T as an input produces the position variable as an output.

The derivative with respect to the x path variable is:

$$\frac{δ L}{δ x}$$

Define the derivative LaGrangian with respect to the (dependent) path variable x, which must be equal to the derivative with the independent variable t of the partial of ΔL with respect to the derivative of the path variable x.

$$\frac{δ L}{δ x} = \frac{d}{{d}_{t}} ( \frac{δ L}{δ \dot{x}} )$$

The derivative with respect to the y path variable is:

$$\frac{δ L}{δ y}$$

Define the derivative LaGrangian with respect to the (dependent) path variable y, which must be equal to the derivative with the independent variable t of the partial of ΔL with respect to the derivative of the path variable y.

$$\frac{δ L}{δ y} = \frac{d}{{d}_{t}} ( \frac{δ L}{δ \dot{y}} )$$

The derivative with respect to the z path variable is:

$$\frac{δ L}{δ z}$$

Define the derivative LaGrangian with respect to the (dependent) path variable z, which must be equal to the derivative with the independent variable t of the partial of ΔL with respect to the derivative of the path variable z.

$$\frac{δ L}{δ z} = \frac{d}{{d}_{t}} ( \frac{δ L}{δ \dot{z}} )$$

Again, the independent variable is time (t), and for every period of time, there are different x, y and z coordinates of point P, which will give us our 3D LaGrangian path variable for P.

The full LaGrangian for any P in M-D is:

$$L ( x , y , z , \dot{x} , \dot{y} , \dot{z} , t ) = \frac{1}{2} m ( {x}^{2} + {y}^{2} + {z}^{2} ) - U ( x , y , z )$$

Where Fx generalized force is:

$$\frac{δ L}{δ x} = - \frac{δ U ( r )}{δ x} = Fx$$

And Fy generalized force is:

$$\frac{δ L}{δ y} = - \frac{δ U ( r )}{δ y} = Fy$$

And Fz generalized force is:

$$\frac{δ L}{δ z} = - \frac{δ U ( r )}{δ z} = Fz$$

Note that this approach is invariant to the coordinate system used (Cartesian, Polar or otherwise). By choosing the LaGrangian that can be defined as T-U, a path for P can be selected that conforms to Newton's second law where:

$$The generalized force = \frac{d}{dt} ( The generalaized momentum )$$

$$\frac{δ L}{δ x} = \frac{d}{dt} ( \frac{δ L}{δ \dot{x}} ) = \frac{d}{dt} ( m \dot{x} ) = \frac{d}{dt} ( generalized momentum )$$

Newton's second law Fx=m\ddot{x}

And

$$\frac{δ L}{δ y} = \frac{d}{dt} ( \frac{δ L}{δ \dot{y}} ) = \frac{d}{dt} ( m \dot{y} ) = \frac{d}{dt} ( generalized momentum )$$

Newton's second law Fy=mÿ

And

$$\frac{δ L}{δ z} = \frac{d}{dt} ( \frac{δ L}{δ \dot{z}} ) = \frac{d}{dt} ( m \dot{z} ) = \frac{d}{dt} ( generalized momentum )$$

Newton's second law Fz=m\ddot{z}

Referring again for FIG. 3, at 330, the processor(s) in the system/device generate end tooth positions based on the orthodontic data set which initially includes data defining the initial/current position of the teeth in the dentition.

In some embodiments, the processor(s) generate the end tooth positions based at least on part on one or more of the functions described above and herein. In some embodiments, the end tooth positions are generated such that the treatment can facilitate movement of the teeth to these end positions through simultaneous counterbalanced moments. In some embodiments, the outcome is tooth movement in the form of symmetrical counterbalanced three dimensional (3D) rotations on every tooth around its center of rotation at or within a defined threshold to its root apex. In some embodiments, the defined threshold is 3 mm.

In some embodiments, the processor(s) are configured to treat dental arches individually and combined to minimize the orthodontic problem to a distorted 3-dimensional surface model. In some embodiments, the processor(s) are configured to perform a convex optimization process to convert the initial-state surface models from the orthodontic data set into a maximized convexity surface model. In some embodiments, the initial-state surface models in the orthodontic data set can be converted/minimized from a distorted surface model into its basic constituents. In some embodiments, the convex optimization can be performed by a solver and/or a controlled user interface for receiving controlled inputs to satisfy an objective function. In some embodiments, the objective function can include maximizing or otherwise increasing final-state tongue volume while providing resolution of malocclusion problems simultaneously.

In some embodiments, the processor(s) generate end tooth positions by determining end positions for each individual tooth which maximize (or minimize) the optimization function while respecting the anatomical landmarks and biomechanical/motion restrictions described herein.

In some embodiments, the processor(s) iterates and/or adjusts end tooth positions until the optimization function criteria had found at least one local minimum/maximum.

In some embodiments, the processor(s) utilize a reinforcement learning model based on a reward function with similar components to the optimization function. For example, the reward function can be based on the tongue volume, respect for the biomechanical/motion restrictions, final arch shape and/or the like.

In some embodiments, the processor(s) generate a user interface for receiving input from at least one input device to adjust an initial position of a tooth to a second position. In some embodiments, the user interface is configured to restrict adjustments to the initial position of the tooth to movements which are based on the anatomical landmarks referenced herein, and/or which respect the biomechanical/motion restrictions referenced herein.

In some embodiments, the processor(s) are configured to restrict adjustments of the tooth position by disallowing an adjusted position to be successfully inputted if it violates the movement restriction parameters. In some embodiments, the processor(s) generate signals to communicate to the user that the movement is/was restricted; for example, displaying the tooth snapping back to its initial position, outputting a warning message or sound, displaying the tooth with a different visual indicator (e.g. showing the tooth in red or with an icon to indicate that the tooth is in a position that is or will be restricted).

In some embodiments, the warning output can be outputted while the user input is in progress. For example, if the input to move a tooth involves clicking and dragging, the warning output can be outputted during the dragging process and before the user input device is released to select a final desired position.

Based on the controlled inputs, the orthodontic data set and/or the (semi-) automatic processes, the processor(s) generate end positions for each tooth in the dentition.

It should be understood that references to the end tooth position references the end tooth position for a particular orthodontic plan, and can but may not necessarily represent the final tooth position which may involve subsequent orthodontic plans.

At 340, the processor(s) generate treatment plan data based on the end tooth positions. In some embodiments, generating the treatment plan data includes generating a series of intermediate tooth positions between the initial position of each tooth and its corresponding end position.

In some embodiments, the intermediate tooth positions are generated such that the required movement between a previous position and a subsequent position in the series of positions respects the biomechanical/motion restrictions referenced herein. For example, in some embodiments, the motion between intermediate positions is restricted to crown movements which are possible through rotations about the root apex of the tooth.

In some embodiments, the processor(s) generate signals for outputting at least a portion of the series of intermediate positions. In some embodiments, generating these signals includes generating data from which 3D models for orthodontic appliances can be produced; the orthodontic applications for facilitating physical movement of the plurality of teeth from the initial positions to the final positions.

In some situations, these 3D models can be generated to enable the orthodontic appliances facilitate physical movement of the teeth without engaging with any attachment appliances them.

In some embodiments, the processor(s) generate signals for displaying a visual representation of at least a portion of the series of intermediate positions on a display, or storing data representing at least the portion of the series of intermediate positions on a storage device.

At 350, the treatment plan data is used to produce appliances for facilitating movement of the teeth from their initial positions to their end positions. In some embodiments, a series of aligners are produced with each aligned configured to facilitate movement of the teeth from one position in the series of intermediate positions to a subsequent position in the series.

In some embodiments, the treatment plan data can be used for linear regression of clinical crown mesh data describing dental motion for aligner manufacturing.

In some embodiments, the treatment plan data can be used for linear regression of clinical crown mesh data encoding motion for fixed brackets/wires manufacturing.

In some embodiments, the timepoints of linear interpolation can be selected to produce interpolated arch models.

In some embodiments, the treatment plan data can be used to generate the 3D printing of interpolated arch models.

In some embodiments, the treatment plan data can be used in the direct printing or other manufacture of aligners. In some embodiments the treatment plan data can be used for the production of thermoplastic formed clear appliances.

In some embodiments, treatment plan data can include model data in the form of STL or other data files for representing the arch(es). In some embodiments, the model data can include data for the maxillary arch and for the mandibular arch for each step (sub-step) of the movement plan.

In some embodiments, the model data is provided to a 3D printer or other manufacturing process to produce 3D models of the arch(es) for each step of the movement plan. These models are then used as molds to create aligners or other appliances. In some embodiments, the appliances are created via vacu-forming and/or thermal forming.

These appliances (e.g. aligners) can be sequentially positioned on a patient's teeth to facilitate the movement of the teeth.

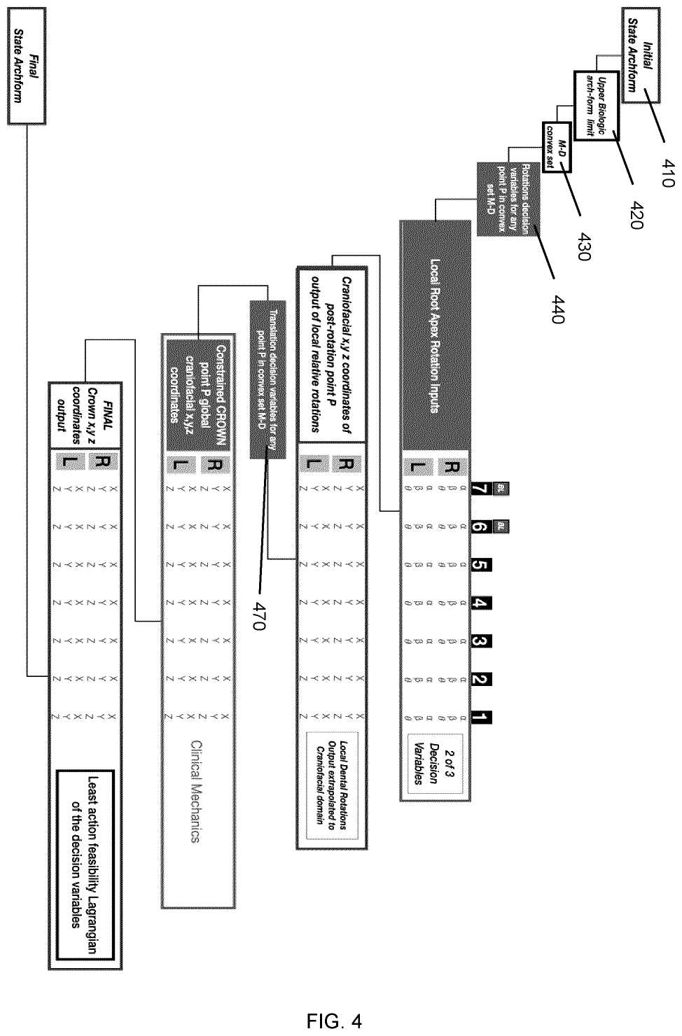

FIG. 4 shows aspects of an example method for generating a treatment plan including the generation of end tooth positions. The aspects of this method or any method referenced herein can be performed by a computing device or system, a clinician, or a combination thereof.

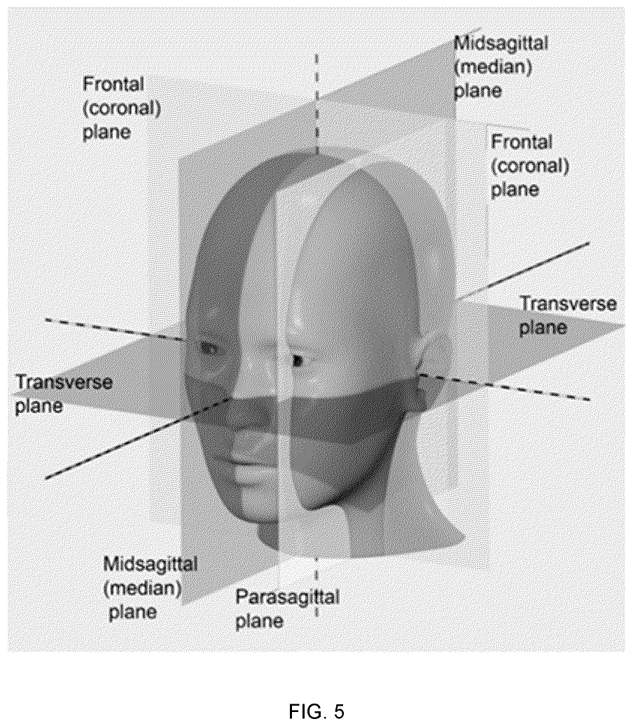

As illustrated in FIG. 5, crown coordinates (x, y, z, or r, theta, z) of any point P within the M-D convex set or dental implement can be measured as a rotation in the three dimensional craniofacial world, the principal craniofacial anatomic planes being Midsagittal, Coronal,

Transverse. While these planes and coordinates are referenced herein, in some embodiments, the processor(s) can be also be implemented to perform the methods described herein using any other suitable coordinate system (e.g. polar coordinates, Cartesian coordinates, etc.).

In some embodiments, the processor(s) can be configured or otherwise operate using and/or referencing the Midline Mid-Sagittal Plane YZ, the Define Coronal Plane XZ, the Transverse Plane XY, the Posterior Perimeter Hyperplane (XZ), the Occlusal hyperplane (XY), and/or the Midline Plane. In some embodiments, these planes comprise at least part of a global cranio-facial domain. In some embodiments, the global cranio-facial domain can be defined by a set of parameters or can be incorporated into geometric calculations. In some embodiments, the global cranio-facial domain can include definitions of global constraints.

As described herein, the orthodontic data set which includes tooth data in the initial state of the archform is accessed, received, loaded or otherwise obtained 410.

In some embodiments, the method for generating the treatment plan starts with the upper arch before the lower arch. This is in contrast to traditional processes which start with the lower arch.

At 420, the width of the upper biologic arch-form is defined by selecting two points on the molar. In some embodiments, the points are selected on the terminal molars. In some embodiments, the points are selected on the second molars. In some embodiments, the points are selected on a distal aspect of the molars.

In some embodiments, by defining the end archform based on the initial width between points on the left and right molars, the end positioning of the teeth respects the patient's natural biologic archform limit.

At 430, in some embodiments, the processor(s) are configured to define the mesial (MCP) and distal contact points (DCP) on the surfaces of each crown. In some situations, the contact points define potential contact points where a particular tooth contacts or is to contact its neighbouring teeth. In some embodiments, the processor(s) configured to define these based on determine contact points from the surface modals/data in the orthodontic data set. In some embodiments, the processor(s) are configured to provide a user interface displaying portions of the dentition via which inputs identifying contact points or adjusting previous/determined contact points can be received.

In some embodiments, the processor(s) convert the contact points for each tooth into a line segment to form an M-D line segment data set. In some embodiments, the line segment data set forms a convex set which can define an upper archform and lower archform through M-D line segment integration.

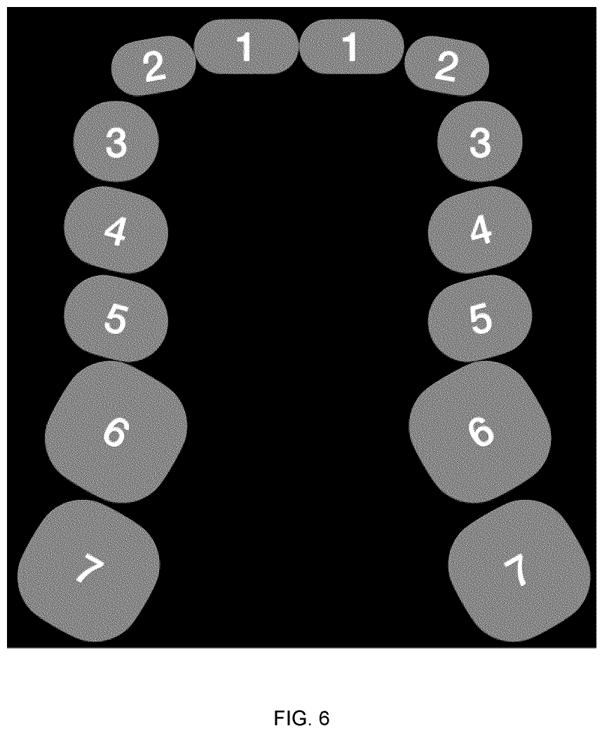

In some situations, by simplifying the complex surface into line segments, the computational complexity of the search space for determining the final tooth positions can be reduced. In some situations, the line segment data can be used to generate planar or cross-sectional representative areas of the teeth as illustrated in FIG. 6. In some embodiments, the line segments can be used to represent the teeth for end position movement as simplified objects such as spheres or other shapes with simplified surfaces and potential contact points.

In some embodiments, the centers of rotations of the M-D convex sets are defined. In some embodiments, the centers of rotations are defined to be at or proximal the root apex of each tooth. In some embodiments, the centers of rotations are defined to be within a threshold distance of the root apex. In some embodiments, the threshold can be 2 or 3 mm.

In some embodiments, the root apex point for at least one molar of the plurality of teeth is at a distal pivot archform point as viewed on the transverse plane.

At 440, the end positions of the teeth are defined. From the posterior teeth to the anterior, the teeth are rotated to align the M and D points of the dental arch. This is in contrast to the traditional aligner approach of starting from the anterior teeth.

In some embodiments, the corresponding left and right tooth in the arch are moved to an end position before moving the next anterior pair of teeth. e.g. the left and right second premolars are moved to an end position before the left and right first premolars, which in turn are moved to their end positions before the left and right canines.

In some embodiments, a tooth's rotations about the root apex are limited to at most 2 of 3 axes of rotations per M-D convex set. In some embodiments, the posterior movement of the most distal coordinate on the clinical crowns of the terminal molars are limited. In some embodiments, this coincides with a clinical distal archform pivot point.

In some embodiments, the relative positions of the upper and lower second molars are final arch-width determinants.

In some embodiments, the teeth are rotated about their centers of rotation to align the M and D points of adjacent teeth along the dental archform.

In some embodiments, the processor(s) generate the end tooth positions with little to no user input. In some embodiments, the processor(s) can generate the end tooth positions following aspects of the methods referenced herein. In some embodiments, the method(s) may:
Use Convex Optimization of the Objective Function of tongue volume as represented in the transverse planar archform area
Use individual crown anatomy especially that of contact points and contact areas to reconstruct the collapsed dental archform under the predetermined constraints and limits
Apply convex optimization mathematics for the objective function of tongue volume/archform space maximization, governed by dentofacial constraint variables
Minimize the stationary moment functions at individual teeth at predetermined t intervals
Use LaGrangian mechanics to compute the LaGrangian-Euler synchronized paths of crown coordinates that satisfy any final or incremental VTO state and adheres to Hamilton's least action principle in order to produce the best set of 28 LaGrangian synchronized paths of crown coordinates, working for the objective function.

Apply LaGrange-Euler Equation to get the crown coordinates given a selected t point, and/or
Input into Aligner vendor linear interpolation for final selection of independent variable t points, and the addition of any clinician modifications to the final prescribed tongue-volume/archform-area optimization movement.

In some embodiments, the convex optimization of final state paths includes:
For any point within M-D line segment, define 3D orthodontic motion paths (roulettes) as hyperbolic convex paths of least action derivation
Action is the counterbalanced synchronized forces/moments operating on the dental arch components (represented by M-D sets) under predetermined constraints, measured as action at any time t acting on a point within the M-D convex set
The objective function is achieving archform area maximum convexity maintaining least action or stationary action optimization of M-D points/convex set's X,Y,Z global coordinate changes with preset maximum limits
Optimizing 2 of 3 derivates of local rotations using LaGrangian or Hamiltonian mathematical hyperbolic geometry.
Solving for optimized convex combinations (x,y,z) of points within the M-D points/convex set of individual teeth
Define Maximum vertical Z LIMIT for each Archform, a positive semidefinite

Maximize the positive semidefinite transverse y derivative, Objective function determinant) Rx maximum, with a max Limit
Minimizing positive semidefinite x A-P derivative, Objective function determinant) Ry minimum limit
Modify Convex local combinations (Rx,Ry,Rz) or (r, θ, z or ϕ) to convert the Convex hull of the M-D line segments integral into a convex set
Optimize M-D points motion path and combinations (Rx,Ry,Rz) to create a convex set
   i. Convex cones search for least action path of two of three hyperbolic functions treated as approximations of least action Rx,Ry,Rz or r, θ,z or ϕ.
   ii. Positive semi-definites of local combinations of Rx(min), Ry (max), Rz(limit), producing Mesial and distal contact points spherical or parabolic LaGrangian paths of least action (applies strictly to any point within M-D convex set)
      1. ΔRx towards a minimized maximum in terms of objective function
      2. ΔRy towards a maximized minimum in terms of objective function
      3. ΔRz towards a minimized maximum in terms of objective function
Define Rz feasible path of MD contact points starting from D point of terminal molar working towards the next M-D point in the sequence ending at the midlines.to the anterior teeth, in increments of 1 degree, optimize for Objective Function
Minimal element approach applied to x coordinate a function of −Ry
Maximum element approach applied to y coordinate a function of +Rx
Optimum elements approach applied to z coordinate a derivative function of Rz, Ry and Rx+higher order linear corrections of +−1-2 mm per tooth Angular velocity limit applied to determine t intervals local (tooth) then Archform linear translations (IOE) with LIMITS
Output Final state integration of M-D convex sets global positions
Linearizing the LaGrangian paths of A between Initial state and final state

In some embodiments, some or all aspects of generation of the end tooth positions can be performed by a clinician such that the limitations and methodologies referenced herein are satisfied.

In some embodiments, the processor(s) in the system/device can facilitate the generation of the end tooth positions by a clinician. In some embodiments, the processor(s) control inputs for defining tooth movements such that they comply with the functions and/or biomechanical rules described herein associated with the tooth.

In some embodiments, this includes the processors generating a user interface for receiving, from at least one input device, at least one input to adjust an initial position of a tooth of the plurality of teeth to a second position. For example, in some embodiments, the user interface is configured to restrict the adjustments to the initial position of a tooth to adjustments having a center of rotation substantially about the root apex point of the tooth.

In some examples, the input can be received when a keyboard input, such as a shortcut key or an entered data value, is received, when a menu option is selected, or in any other manner. In some examples, an input can be received through an interaction with or via an image or visual representation of at least a portion of the dental data set. For example, a user can click and/or drag a cursor, provide keyboard inputs, or touch and swipe a touchscreen, over a tooth to adjust its position or orientation. In some examples, an input to edit dental information can include any one or combination of clicks, gestures, keyboard or shortcut inputs. In some examples, an input can include audio commands.

Receiving an input to edit dental information with or via a visual representation may, in some examples, include receiving an input via a slider, selection box/list or other parametric adjustment interface element.

In some embodiments, the processors are configured to control or restrict the movement of the tooth in response to the input from the input device. In some embodiments, the processors can be configured to restrict the movement of the tooth in response to the input from the input device to a motion which respects the restrictions of motion about the center of rotation proximal the root apex.

Parameters included in the orthodontic data set/treatment plan data can be adjusted based on the received input. In some examples, by iteratively receiving inputs and adjusting parameters, a data can be updated to reflect the desired movement of the teeth.

In some embodiments, the user interface is configured to restrict the adjustments to the initial position of the first tooth to adjustments having a centre of rotation substantially about the root apex point of the first tooth by displaying user interface elements representing the first tooth position moving substantially about the root apex point of the first tooth in response to received inputs.

In some embodiments, the processor(s) generate a user interface which allows a tooth position to be intermediately translated or rotated in directions or along axes which are not restricted to adjustments having the centre of rotation substantially about the root apex point of tooth.

In some embodiments, the processor(s) generating signals for the user interface to restrict the acceptance of a tooth position defined by the inputted adjustments as a final position for the tooth when a total adjustment of the tooth from the initial position does not have a centre of rotation substantially about the root apex point of the tooth.

In some embodiments, the processor(s) restrict the movement of teeth until a previous tooth in the posterior to anterior order has already been adjusted or confirmed to be in a desired position.

In some embodiments, the processor(s) restrict the movement of a first tooth to a molar such as the second molar or a rear or terminal molar.

In some embodiments, the processor(s) generate, using the orthodontic data set, a user interface for receiving, from at least one input device, a tooth selection input for selecting the first tooth for which the initial position is to be adjusted; wherein the user interface is configured to restrict the tooth selection of the first tooth to a molar.

In some embodiments, the processor(s) generate, using the orthodontic data set, a user interface for receiving, from at least one input device, a tooth selection input for selecting a subsequent tooth for which the initial position is to be adjusted, and the user interface is configured to restrict the tooth selection for the subsequent tooth to be an anteriorly adjacent tooth to a previously adjusted tooth.

In some embodiments, the processor(s) generate, using the orthodontic data set, a user interface for receiving, from at least one input device, a tooth selection input for selecting a subsequent tooth for which the initial position is to be adjusted; wherein the user interface is configured to restrict the tooth selection such that adjustment of the plurality of teeth progresses from the posterior to the anterior of the dentition.

In some embodiments, the user interface is configured to restrict the tooth selection of a subsequent tooth in the anterior direction until the corresponding left/right tooth has been adjusted.

In some embodiments, the user interface is configured to automatically select a next tooth in the dentition which respects the order described herein.

In some embodiments, the user interface is configured to restrict the tooth selection such that adjustment of a tooth of the plurality of teeth situated in an upper jaw of the dentition is adjusted before a corresponding tooth in the lower jaw of the dentition.

In some embodiments, the user interface is configured to restrict the adjustments to the initial position of the first tooth to adjustments based on the biologic archwidth.

At 470, in some embodiments, the end positions of the teeth after the rotational movements can be adjusted through constrained translations of individual M-D sets for leveling of the entire archform (e.g. for bite correction).

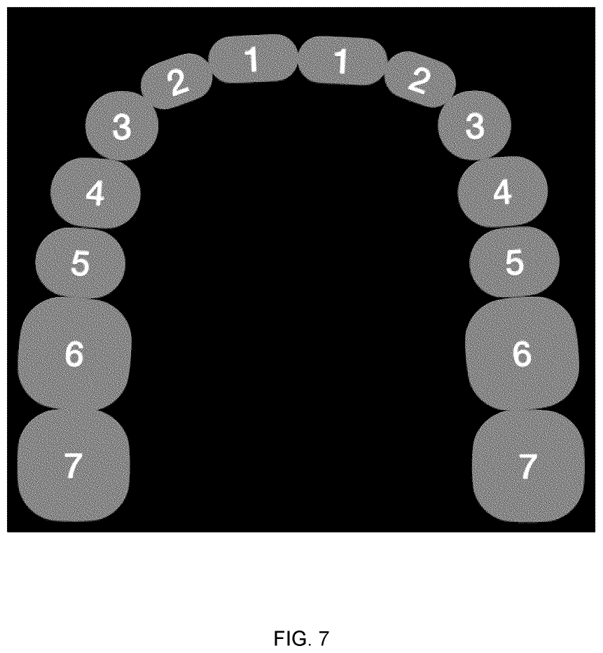

FIG. 7 shows an example of the final tooth positions for the teeth in the initial dentition of FIG. 6.

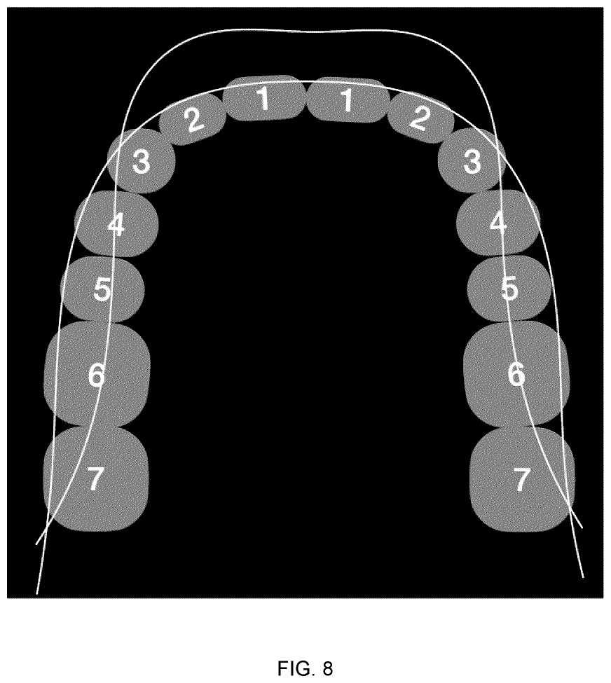

FIG. 8 shows the same example along with the before and after archforms.

In some of the above embodiments or otherwise, the methods referenced herein can utilize the following parameters and/or steps. In some embodiments, these parameters are enforced by the processor(s):
1. Linear changes are calculated in separation and parallel to rotation interpolation resulting a cycloid interpolation (a form of parabola represented using hyperbolic geometry, LaGrangean mechanics or Hamiltonian mechanics for convex optimization of tongue volume
2. No linear velocity factoring
3. An optimized substitute of linear regression of dental motion
4. Conversion of baseline local dental spherical behaviour to a global hyperbolic path minimization according to Fermat's principle of least time, taking into account human mechanical functional environment
5. Action: Counterbalanced Minimized Positive semidefinite Moment Vector acting under stationary action constraints for orthodontic tooth movement
6. Work: Theta changes at a near constant rate (linear) with respect to time
7. Stationary Action Principle or Least Time Principle
8. Define Cranio-facial Global Coordinate System XYZ
   8.1.Define Midline Mid-Sagittal YZ
   8.2.Define Coronal Plane XZ
   8.3.Define Transverse Plane XY
   8.4.Posterior Perimeter Hyperplane (XZ)
   8.5.Define Occlusal hyperplane (XY)
   8.6.Define Midline Plane
   8.7.Define Global constraints
9. Define Dental Domain
   9.1. Define Mesial contact-points
   9.2.Define Distal contact-points
   9.3.Define MD convex sets [CSET] for multi rooted molars
   9.4.Define MD convex set [CSET] for all single rooted teeth
   9.5.Normal @ FA point in MD hyperplane is convex and affine
   9.6.Normal @ FA point in MD Half-space is convex not affine
   9.7.Define Local Dental constraints
   9.8. Define local centre of rotations as a default transformation to estimated points within the region of root apices +2-3
   9.9. Contained Local behaviour to teeth will now be extrapolated to some Global Property of its position
10. Define Initial State
   10.1.Define MD normal
   10.2.Define initial state dental arch form as the integral of the MD lines
   10.3.Define Dental form terminal maximas [XYZ coordinates] fixed
   10.4.Define terminal tooth pivot point
   10.5.Define X coordinate of terminal tooth pivot point=Dental arch Pivot point −X up to a maximum
11. Define Optimized Final state
   11.1. Define 3D orthodontic motion (roulette) as hyperbolic convex paths of least action derivation for any point within CSET
   11.2. For the objective function of Archform maximum convexity maintaining least action or stationary action optimization of CSET & crown X,Y,Z global coordinate changes with preset limits
   11.3. Minimizing distance contact point to arch pivot point
   11.4. Optimizing Theta of 2 out of 3 derivates of local rotations (LaGrangian or Hamiltonian mathematical solution for Hyperbolic Geometry)
   11.5. Solving for optimized convex combinations of individual teeth:
      11.5.1. Maximum vertical Z LIMIT of dental arch
      11.5.2. Maximizing the positive semidefinite Transverse X, Objective function determinant) y maximum, Limit
      11.5.3. Minimizing positive semidefinite Y (IPR, [Clinician Decision Input, IPR], Objective function determinant) a minimum limit
   11.6. Modify convex combinations α,γθ to convert the convex hull of 32 arch form points into a convex set
   11.7. Optimize MD point's motion path and combinations (α,γ,θ) to create a convex set
      11.7.1. Convex cones search for least action path of two of three hyperbolic functions treated as spherical functions α,γ,θ solving for the third
      11.7.2. Positive* semi-definite of α (min), γ (max), θ(lim) of Mesial and distal contact points spherical or parabolic path (applies strictly to any point between MD points)
      11.7.3. Δα towards a minimized maximum
      11.7.4. Δγ towards a maximized minimum
      11.7.5. Δθ towards a minimized maximum
   11.8. θ feasible hyperbolic path of MD contact points [Clinician Decision Input, IPR LIMITS] and Objective Function
   11.9. Minimal element approach applied to Y a function of −α
   11.10.Maximum element approach applied to X a function of +γ
   11.11. Optimum elements approach applied to Z a derivative function of θ, γ and α+higher order linear corrections of +−1-2 mm per tooth [Clinician Decision Input, local or global linear translations positive semidefinite with LIMITS]
   11.12. Variable angular velocity limit applied to the resultant Theta [Clinician Decision Input, first local (tooth) then global linear translations (IOE) as positive semidefinite with LIMITS]
12. Output Final state integration of CSET global positions
13. Linearizing of 3 rotations of rigid body rotation as a hyperbolic linear integration
14. Can be used for linear regression of lower order clinical crown mesh data describing motion for aligner manufacturing
15. Can be used for linear regression of lower order clinical motion data describing motion for fixed brackets/wires manufacturing

In some embodiments, aspects of the present application can provide a least vector simultaneous aligner mechanics, treatment planning and treatment execution system. In some situations, aspects of the present methods and systems can create aligners and/or other applications which may effectively cause desired tooth outcomes with: no attachments, no IPR, no staging, simultaneous mechanics, fewer than 25 aligner in each bout, no bite jumps or attempts for bite correction (inter-arch mechanics), variable interval execution and/or LaGrangian/Hamiltonian orthodontic mechanics. Any one or more of these features may provide notable technical improvement(s) over previous aligner approaches.

In some embodiments, a method for generating an orthodontic treatment plan, can be based on one or more of the following:

In some embodiments, the method includes an intra-arch mechanics planning phase. In some embodiments, with at least aspects of the tools and/or processes described herein, or otherwise, this can include orthodontic treatment VTO planning, tracking the rotations locally and the translations globally of any P point (supposing that point P is any point within a given M-D convex set). These process(es) and rules are defined by the processor(s) and in some embodiments, may be provided with a user interface to facilitate them with inputs received from one or more input devices.

In some embodiments, aspects of the example method can produces a globally interpreted simultaneous orthodontic motion plan for all P points, to be used for orthodontic treatment for the objective function of tongue volume maximization as a solution to dental arch collapse. Teeth are considered as constrained rotating bodies; therefore it helps to present and treat local dental coordinate system as a polar one with the center of rotations as described above. Local dental rotations are extrapolated to the craniofacial coordinate system as some change in x,y,z values of any point within M-D convex set of the respective tooth. Any linear dental translations can be viewed as x,y,z translations of any point within M-D convex set of the respective tooth, such linear translations require special treatment compared to local rotations, and in some embodiments are accounted for after rotations and during treatment execution as they require complimentary clinical mechanics such as inter-arch mechanics for:
Inversion of a moment action
Management of clinical moments anchorage
Extrusion of dentition to occlusal hyperplane
Antero-posterior dental arch relative changes
Transverse dental arch relative changes
Growth modification
Surgical planning

In some embodiments, the method includes: building rotations first, and adding translations later after full arch rotations are completed. In some embodiments, the processor(s) are configured to display interfaces and/or instructions in a sequence to enforce this order.

In some embodiments, the processor(s) are configured to start and/or otherwise facilitate the VTO process with upper arch, perform the following actions in pairs starting from posterior to anterior on the Right and Left side symmetrically for both dental arches:

Second molars. In some embodiments, the processor(s) use the existing transverse dimension y (transverse axis) to establish the patient's biologically determined dental arch width as a constraint, this is the starting point for the upper arch development.

In some embodiments, the processor(s) define: Local Rotations inputs, rotation constraints and semi-definites:
1. Rz (main variable) local Rotation: rotation Rz of terminal molars is changed, to the extent necessary as a positive semidefinite (mesial out), a tangent to FA point should not exceed 90 degrees to the Archform Posterior Perimeter Hyperplane (YZ).
2. Ry Minimal local rotation around y, parallel hyperplanes tangent to M or D points should not exceed 90 degrees to the Occlusal hyperplane (XY).
3. Rx Minimal local Rotation around x, a positive semidefinite, where a tangent hyperplane to FA point should not exceed 90 degrees to the Occlusal hyperplane (XY).

In some embodiments, the processor(s) define: Global translations constraints and semi-definites:
1. Tx Minimal global translation in x (Antero-posterior axis): the current intermolar width of terminal molars are not changed, keeping their distal aspect at a minimum in x axis.
2. Ty Minimal global translation in y (transverse axis): Rotate 7 s (mesial out) around the Distal Pivot Archform point, keeping the distal aspect at minimal change in y axis.
3. Tz Minimal global translation in z (vertical axis): current vertical position of terminal molars are not changed, except as a positive semidefinite if for allowing occlusal contact of functional cusps and the contribution to a flat occlusal plane.

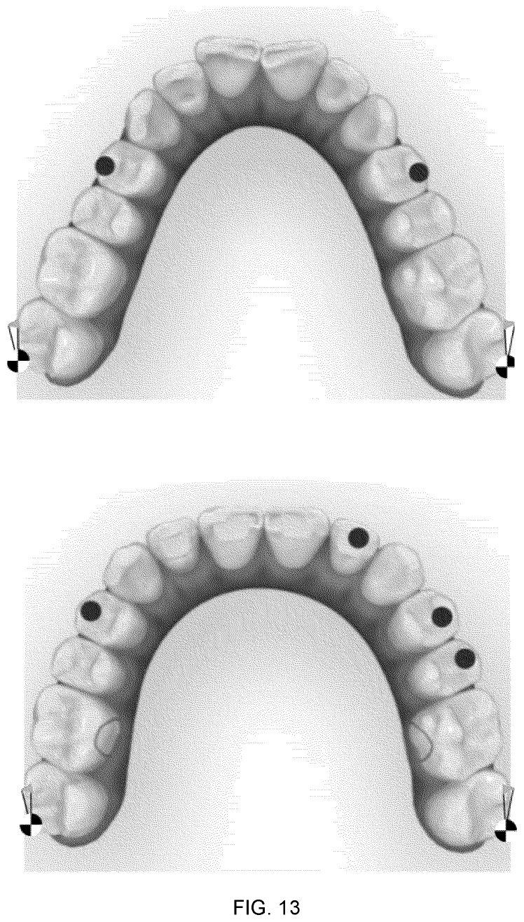

FIG. 13 which shows example visual representations of a portion of a dentition showing some of these parameters.

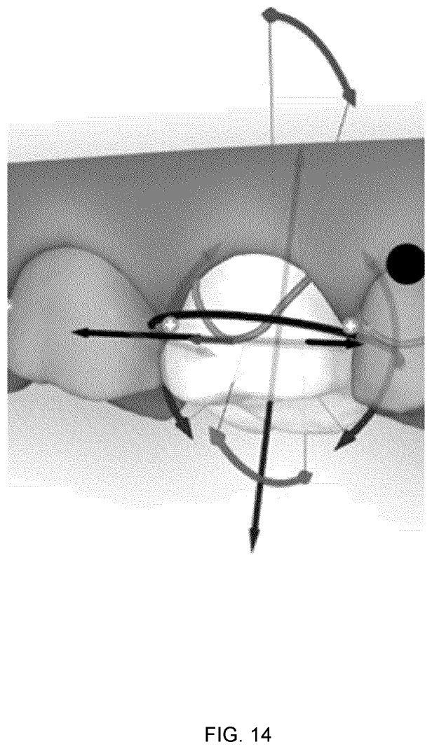

First Molars and the rest of the dentition. In some embodiments, the processor(s) provide a user interface to control/enable the following steps and/or parametric restrictions: Build a rotation of P around the Distal Pivot Archform point minimizing distance between 7 M and 6 D M-D points. Rotate 6 s (mesial out) to achieve the best contact area fit minimal distance between 7 M and 6 D and maximizing the z coordinates of P to Archform maximums to contribute to flattening of the final state occlusal plane. The magnitude of rotation and translation of the crown on the transverse plane is determined based on the initial state (current tooth position) and any final state (VTO) position of 7 M. This can be accomplished for example using treatment planning solutions as illustrated with some of the example controls below, and FIG. 14:

### First, Local Rotations Inputs, Rotation Constraints and Semi-Definites

1. Rz local Rotation around Rz: Change the rotation Rz of 6 s, to the extent necessary as a positive semidefinite to minimize distance between 7 M and 6 D, a hyperplane or tangent at FA point should not exceed 90 degrees to the Archform Posterior Perimeter Hyperplane (YZ).
2. Ry local Rotation around y: Change the rotation Ry of 6 s, to the extent necessary as a positive semidefinite to minimize distance between 7 M and 6 D, parallel hyperplanes tangent to M or D points should not exceed 90 degrees to the Occlusal hyperplane (XY).
3. Rx local Rotation around x: Change the rotation Rx of 6 s, to the extent necessary as a positive semidefinite to minimize distance between 7 M and 6 D, a tangent hyperplane to FA point should not exceed 90 degrees to the Occlusal hyperplane (XY).

### Second, Global Linear Translations Input Constrains and Semi-Definites

4. Tx global translation changes in x coordinate of P beyond what above rotations produce mesial or distal linear change to simulate interarch mechanics.
5. Ty global translation changes in y coordinate of P beyond what above rotations produce Buccal or lingual linear change to simulate interarch mechanics.
6. Tz global translation changes in z coordinate of P beyond what above rotations produce occlusal or apical linear change to simulate interarch mechanics and normal vertical development. The current vertical position of molars are not changed, except as a positive semidefinite only if:
   a. For allowing occlusal contact of functional cusps
   b. The creation of a flat occlusal plane
   c. Clinician Translations corrections for inter-arch mechanics

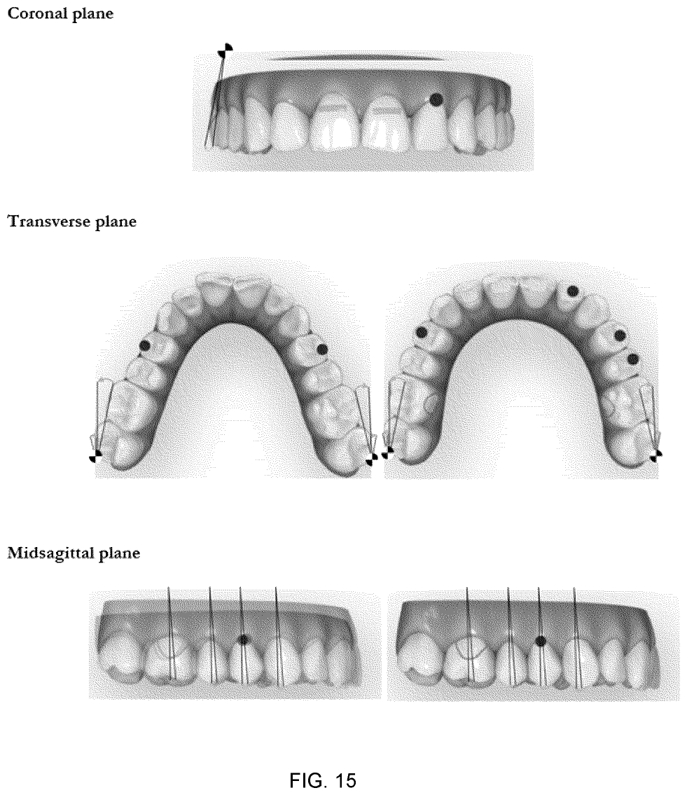

FIG. 15 shows example visual representations of a portion of a dentition with movement annotations some of these rotations.

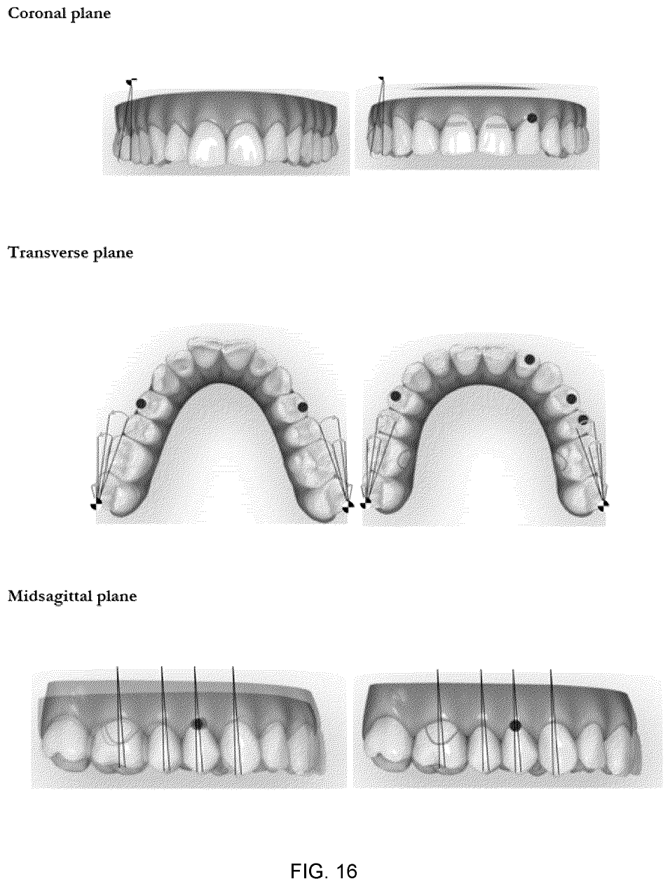

Next, FIG. 16 shows example visual representations of a portion of a dentition with additional movement annotations with respect to Second Premolars #1.5 & #2.5.

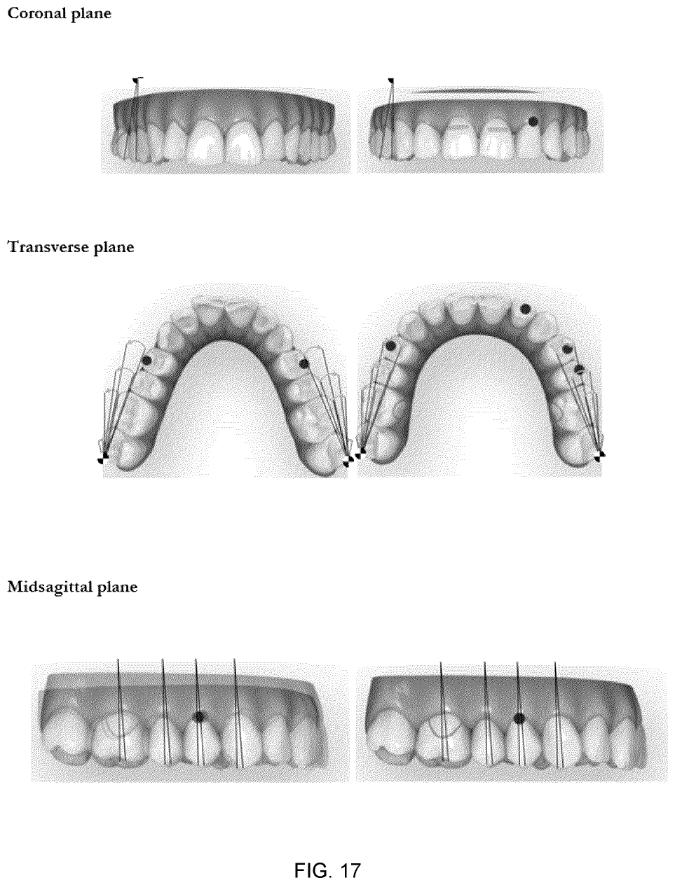

Next, FIG. 17 shows example visual representations of a portion of a dentition with additional movement annotations with respect to First premolars #1.4 & #2.4.

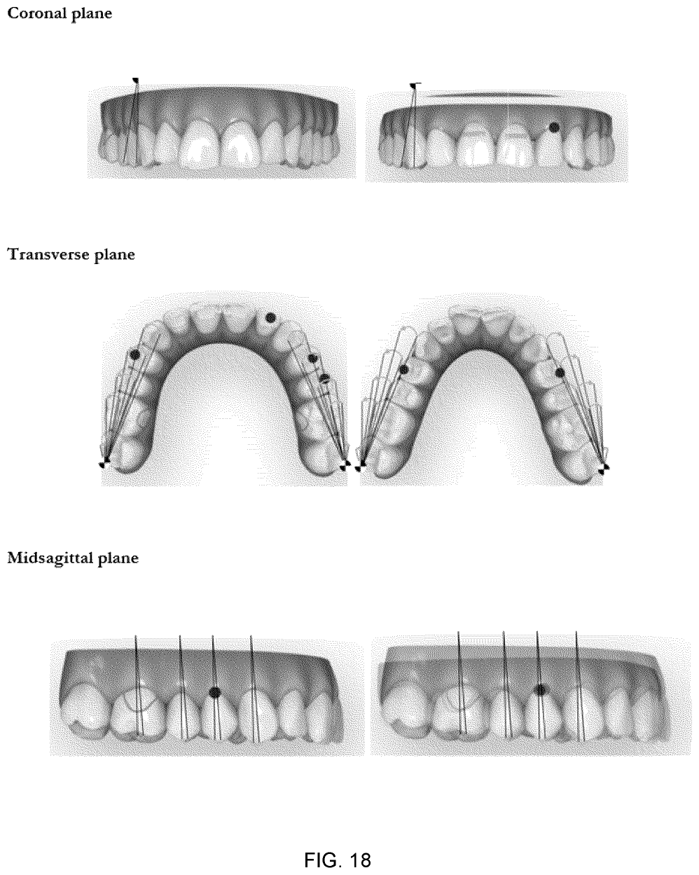

Next, FIG. 18 shows example visual representations of a portion of a dentition with additional movement annotations with respect to Canines #1.3 & #2.3.

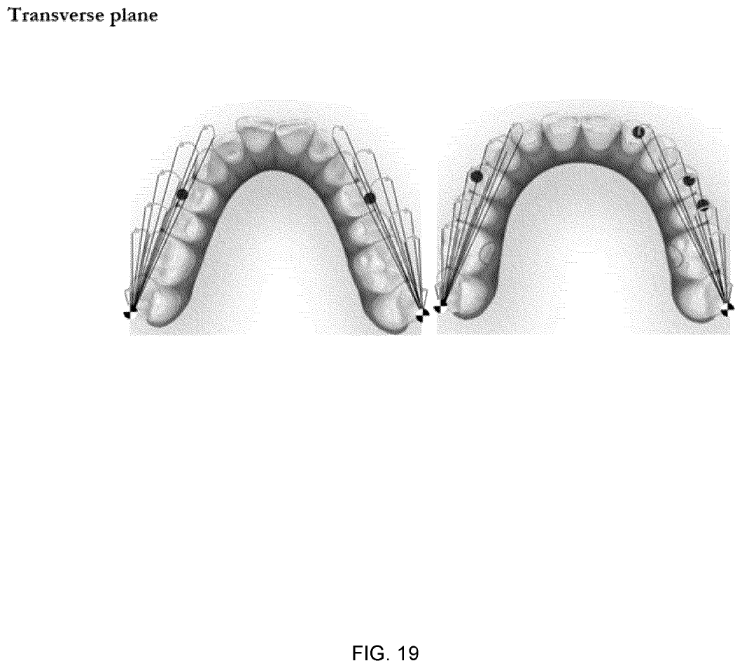

Next, FIG. 19 shows example visual representations of a portion of a dentition with additional movement annotations with respect to Lateral incisors #1.2 & #2.2.

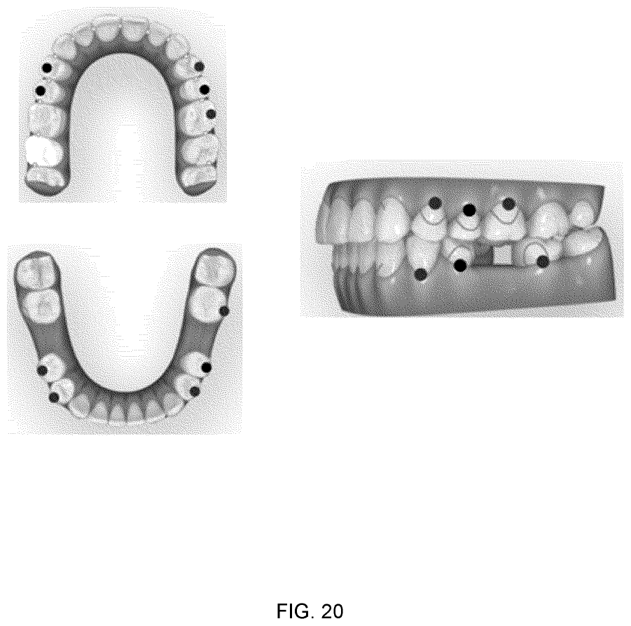

In some embodiments, the method includes analyzing the orthodontic data set as illustrated in the example visual representations in FIG. 20 with respect to the arch symmetry, and adjust the archform to establish arch symmetry.

In some embodiments, the method proceeds to the lower arch, performing the same actions starting from posterior to anterior on Right and Left side symmetrically. In some embodiments, the process starts with the 7's moving forward towards the patient's midline.

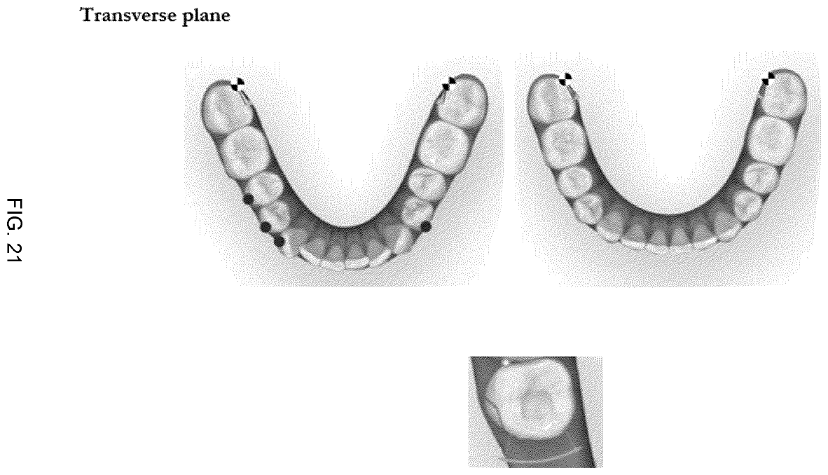

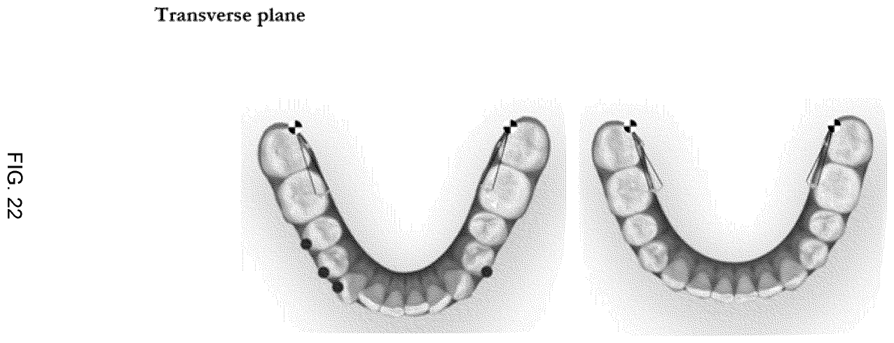

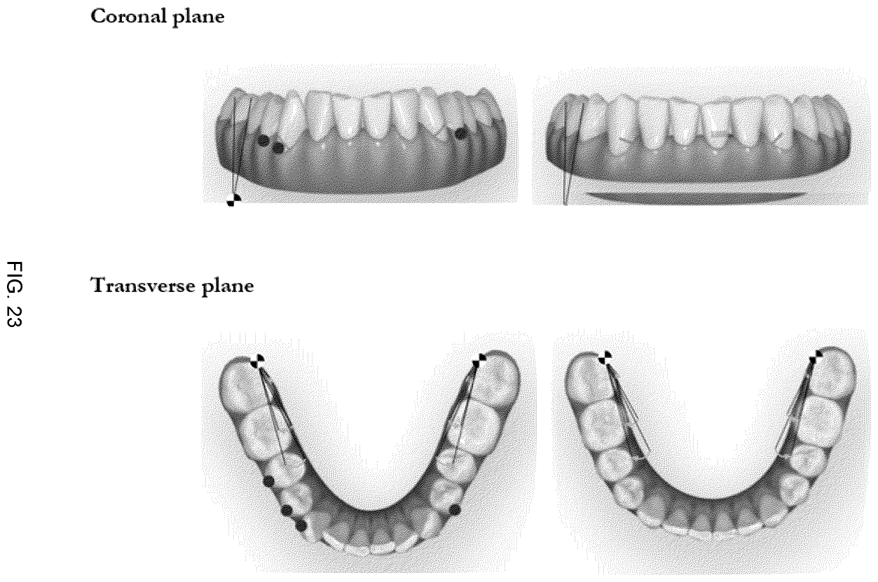

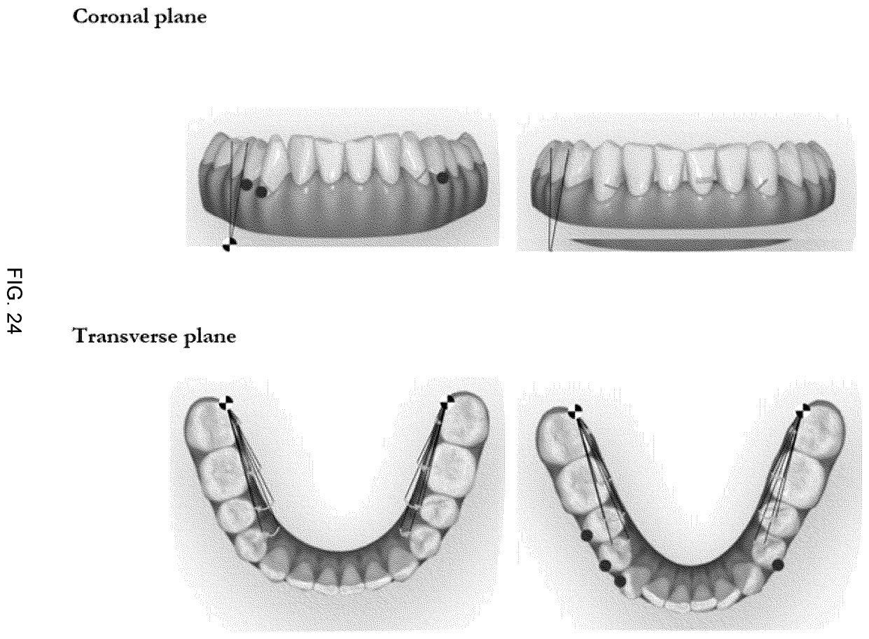

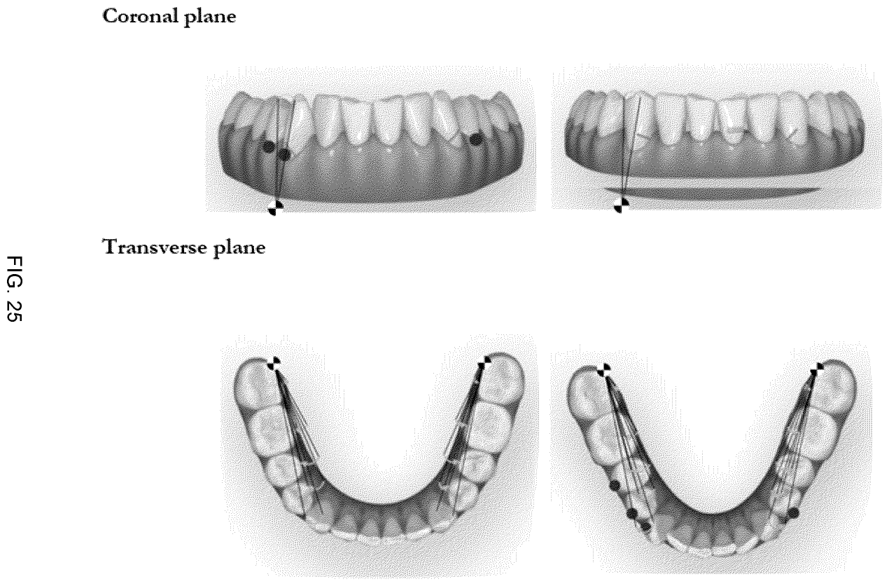

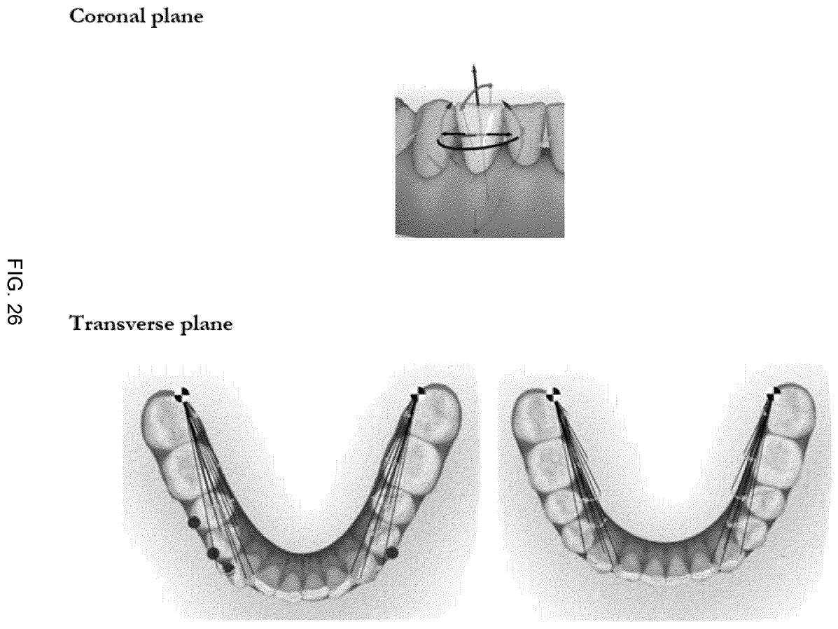

Example movement annotations following this sequence are illustrated in the example visual representations of the lower arch:
FIG. 21—Second molars #3.7 & #4.7
FIG. 22—First molars #3.6 & #4.6
FIG. 23—Second premolars #3.5 & #4.5
FIG. 24—First premolars #3.4 & #4.4
FIG. 25—Canines #3.3 & #4.3
FIG. 26—Lateral incisors #3.2 & #4.2

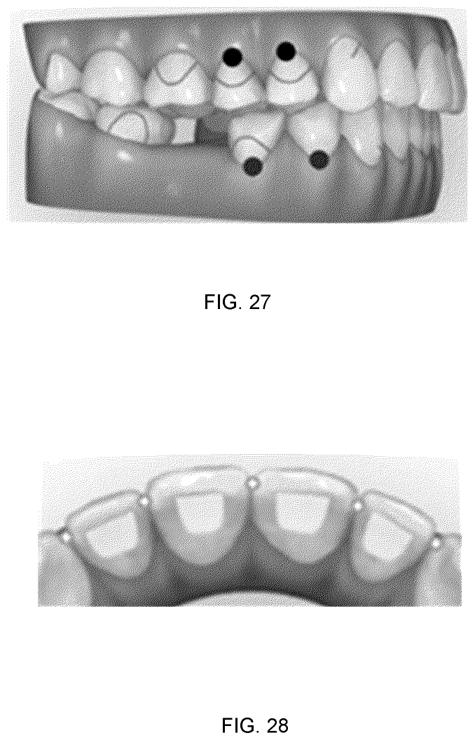

With reference to FIG. 27, in some embodiments, the method can include (when necessary), examining arch symmetry, and applying the interarch relative linear translations, which in turn dictate inter-arch mechanics used during treatment execution.

With reference to FIG. 28, in some embodiments, the method can include (when necessary), analyzing inter arch mechanics. In some embodiments, the anterior teeth are defined to have anterior bite turbos to activate full arch suctional forces. In some embodiments, mechanics aimed at changing the relationship between upper and lower arches are established, and lower arch coordination with upper can be examined:
1. Check advanced mechanics requirements
   a. Elastics cut outs
   b. Mandibular advancement TB
   c. Deciduous teeth virtual geometry
2. Check for and resolve heavy occlusal contacts, ideally occlusal contact points between upper and lower teeth remains unchanged or minimally increased across the entire dental arch
3. Tooth size discrepancy restorative management
4. Elastics for bite correction.
   Short Cl 2
   Reg Cl2
   Short Cl3
   Reg Cl3
   TTB Cl3 NT
   TTB CL2 NT
   TTB X-bite
   TTB (custom)

With reference to FIG. 28, in some embodiments, the method can include (when necessary), analyzing inter arch mechanics. In some embodiments, the anterior teeth are defined to have anterior bite turbos to activate full arch suctional forces. In some embodiments, mechanics aimed at changing the relationship between upper and lower arches are established, and lower arch coordination with upper can be examined.

In some embodiments, the treatment plans, aligners and/or other appliances can be generated with different treatment execution/wear patterns, for example: Full-time with acceleration, Full-time weekly, Full-time biweekly, Part-time biweekly, and/or Part-time monthly.

While the foregoing invention has been described in some detail for purposes of clarity and understanding, it will be appreciated by those skilled in the relevant arts, once they have been made familiar with this disclosure, that various changes in form and detail can be made without departing from the scope of the invention. The invention is therefore not to be limited to the exact components or details of methodology or construction set forth above. Except to the extent necessary or inherent in the processes themselves, no particular order to steps or stages of methods or processes described in this disclosure, including the Figures, is intended or implied. In many cases the order of process steps may be varied without changing the purpose, effect or import of the methods described.

1. A computer-implemented method for orthodontic appliances, the method comprising:
obtaining from a storage device or generating a first orthodontic data set representing initial positions for each of a plurality of teeth in at least a portion of a dentition, the first orthodontic data set including a position of a root apex point for each of the plurality of teeth;
generating, using the first orthodontic data set, a user interface for receiving, from at least one input device, at least one input to adjust an initial position of a first tooth of the plurality of teeth to a second position; wherein the user interface restricts the adjustments to the initial position of the first tooth to adjustments having a centre of rotation substantially about the root apex point of the first tooth;
adjusting the data representing the first orthodontic data set based on the at least one input to include data representing a final position of the first tooth;
generating a series of intermediate teeth positions between the initial positions for each of the plurality of teeth represented in the first orthodontic data and final positions for each of the plurality of teeth, the series of intermediate positions comprising at least part of a treatment plan; and
generating signals for outputting at least a portion of the series of intermediate positions.

2. The method of claim 1, wherein generating the signals for outputting at least the portion of the series of intermediate positions comprises generating data from which 3D models for orthodontic appliances can be produced; the orthodontic applications for facilitating physical movement of the plurality of teeth from the initial positions to the final positions.

3. The method of claim 2, wherein the based on the series of intermediate teeth positions, the 3D models are generated which enable the orthodontic appliances facilitate physical movement of the plurality of teeth without engaging with any attachment appliances on the plurality of teeth.

4. The method of claim 1, wherein generating the signals for outputting least the portion of the series of intermediate positions comprises signals for displaying a visual representation of at least a portion of the series of intermediate positions on a display, or storing data representing at least the portion of the series of intermediate positions on a storage device.

5. The method of claim 1, wherein the root apex point for at least one tooth of the plurality of teeth is within 3 mm of a root apex of the at least one tooth in the first orthodontic data.

6. The method of claim 1, wherein the root apex point for at least one molar of the plurality of teeth is at a distal pivot archform point as viewed on the transverse plane.

7. The method of claim 1, wherein the user interface is configured to restrict the adjustments to the initial position of the first tooth to adjustments having a centre of rotation substantially about the root apex point of the first tooth by displaying user interface elements representing the first tooth position moving substantially about the root apex point of the first tooth in response to received inputs.

8. The method of claim 1, wherein the user interface is configured to restrict the adjustments to the initial position of the first tooth to adjustments having a centre of rotation substantially about the root apex point of the first tooth by:
allowing the first tooth position to be intermediately translated or rotated in directions or along axes which are not restricted to adjustments having the centre of rotation substantially about the root apex point of the first tooth; and
generating signals for the user interface restricting the acceptance of a second tooth position defined by the adjustments for the first tooth as a final position for the first tooth when a total adjustment of the first tooth from the first position to the second position does not have a centre of rotation substantially about the root apex point of the first tooth.

9. The method of claim 1, wherein the user interface is configured to restrict the first tooth of the plurality of teeth to a molar.

10. The method of claim 9, wherein the user interface is configured to restrict the first tooth of the plurality of teeth to a second molar or a rear molar.

11. The method of claim 10, comprising generating, using the first orthodontic data set, a user interface for receiving, from the at least one input device, a tooth selection input for selecting the first tooth for which the initial position is to be adjusted; wherein the user interface is configured to restrict the tooth selection of the first tooth to a molar.

12. The method of claim 1, comprising generating, using the first orthodontic data set, a user interface for receiving, from the at least one input device, a tooth selection input for selecting a subsequent tooth for which the initial position is to be adjusted; wherein the user interface is configured to restrict the tooth selection for the subsequent tooth to be an anteriorly adjacent tooth to a previously adjusted tooth.

13. The method of claim 1, comprising generating, using the first orthodontic data set, a user interface for receiving, from the at least one input device, a tooth selection input for selecting a subsequent tooth for which the initial position is to be adjusted; wherein the user interface is configured to restrict the tooth selection such that adjustment of the plurality of teeth progresses from the posterior to the anterior of the dentition.

14. The method of claim 1, comprising generating, using the first orthodontic data set, a user interface for receiving, from the at least one input device, a tooth selection input for selecting a subsequent tooth for which the initial position is to be adjusted; wherein the user interface is configured to restrict the tooth selection such that adjustment of a tooth of the plurality of teeth situated in an upper jaw of the dentition is adjusted before a corresponding tooth in the lower jaw of the dentition.

15. The method of claim 1, comprising determining, using the first orthodontic data set, a biologic archwidth of the dentition

16. The method of claim 15, wherein the biologic archwidth is based on based on a distance between second molars, or a distance between terminal molars.

17. The method of claim 15, wherein the user interface is configured to restrict the adjustments to the initial position of the first tooth to adjustments based on the biologic archwidth.

18. A method for generating an orthodontic treatment plan with a computing device,
inputting, with at least one input device, for each tooth in a plurality of teeth in an orthodontic data set representing at least a portion of a dentition, at least one input to adjust an initial position of each tooth of the plurality of teeth to a second position, wherein the adjustments to the initial position of each tooth has a centre of rotation substantially about the root apex point of the tooth;
generating, the computing device, a series of intermediate teeth positions between the initial positions for each of the plurality of teeth represented in the orthodontic data and final positions for each of the plurality of teeth, the series of intermediate positions comprising at least part of a treatment plan; and
generating signals for outputting at least a portion of the series of intermediate positions.

19. An electronic device for charting dental information, the device comprising: the device comprising:
at least one memory; and
at least one processor configured for performing the method of claim 1.

20. A non-transitory, computer-readable medium or media having stored thereon computer-readable instructions which when executed by at least one processor configure the at least one processor for: performing the method of claim 1.

21. A method for orthodontic appliances, the method comprising:
obtaining or generating a first orthodontic data set representing initial positions for each of a plurality of teeth in at least a portion of a dentition, the first orthodontic data set including a position of a root apex point for each of the plurality of teeth;
generating, using the first orthodontic data set, final positions for each of the plurality of teeth represented in the first orthodontic data set; wherein generating the final positions includes restricting the adjustments to the initial position of each tooth to adjustments having a centre of rotation substantially about the root apex point of the respective tooth;
generating a series of intermediate teeth positions between the initial positions for each of the plurality of teeth represented in the first orthodontic data and final positions for each of the plurality of teeth, the series of intermediate positions comprising at least part of a treatment plan; and
generating data from which orthodontic appliances, which facilitate movement through the intermediate tooth positions, can be produced.

22. The method of claim 21 wherein generating the final positions includes determining end positions for each tooth based on an optimization function.

23. The method of claim 21 wherein generating the final positions includes utilizing a user interface which restricts the adjustments to the initial position of a first tooth to adjustments having a centre of rotation substantially about the root apex point of the first tooth.
